(19) United States
(12) Patent Application Publication
(10) Pub. No.: US 2026/0026445 A1
(43) Pub. Date: Jan. 29, 2026

(54) MODIFIED PLANTS AND SEEDS WITH ENHANCED PHYSIOLOGICAL PERFORMANCE AND ENVIRONMENTAL STRESS RESISTANCE

(71) Applicant: Texas Tech University System, Lubbock, TX (US)

(72) Inventors: Chien Van Ha, Lubbock, TX (US); Lam-Son Phan Tran, Lubbock, TX (US); Luis Herrera-Estrella, Lubbock, TX (US); Monty Christian, Lubbock, TX (US)

(73) Assignee: Texas Tech University System, Lubbock, TX (US)

(21) Appl. No.: 18/993,995

(22) PCT Filed: Jul. 13, 2023
(86) PCT No.: PCT/US2023/027600

§ 371 Date: Jan. 13, 2025

Related U.S. Application Data
(60) Provisional application No. 63/388,878, filed on Jul. 13, 2022.

(51) Int. Cl. A01H 1/06; A01H 1/00

(52) U.S. Cl. CPC A01H 1/06; A01H 1/121; A01H 1/1225

(57) ABSTRACT

Embodiments of the present disclosure pertain to methods of enhancing a physiological performance or environmental stress resistance of a plant or seed by exposing the plant or seed to a composition that includes ethanol, acetic acid, or combinations thereof. Additional embodiments of the present disclosure pertain to modified plants or seeds that demonstrate enhanced physiological performance, enhanced environmental stress resistance, or combinations thereof. In some embodiments, the modified plants or seeds are formed by exposing the plants or seeds to a composition of the present disclosure. In some embodiments, the enhanced physiological performance or environmental stress resistance may occur through inheritable epigenetic modifications in the plant or seed.

### CROSS-REFERENCE TO RELATED APPLICATIONS

This application claims priority to U.S. Provisional Patent Application No. 63/388,878, filed on Jul. 13, 2022. The entirety of the aforementioned application is incorporated herein by reference.

### BACKGROUND

A need exists for crops and seeds with enhanced physiological performance and resistance to environmental stress. Numerous embodiments of the present disclosure aim to address this need.

### SUMMARY

In some embodiments, the present disclosure pertains to methods of enhancing a physiological performance or environmental stress resistance of a plant or seed. In some embodiments, the methods of the present disclosure include a step of exposing the plant or seed to a composition. In some embodiments, the composition includes one or more active ingredients. In some embodiments, the active ingredients include, without limitation, ethanol, acetic acid, and combinations thereof.

Additional embodiments of the present disclosure pertain to modified plants or seeds that demonstrate enhanced physiological performance, enhanced environmental stress resistance, or combinations thereof. In some embodiments, the modified plants or seeds of the present disclosure are formed by the methods of the present disclosure. In some embodiments, the modified plants or seeds are formed by exposing the plants or seeds to a composition of the present disclosure (i.e., a composition that includes one or more active ingredients, where the active ingredients include ethanol, acetic acid, and combinations thereof).

### BRIEF DESCRIPTION OF THE DRAWINGS

FIGS. 1A-1D provide experimental results demonstrating that acetic acid and ethanol treatments enhanced drought tolerance and stimulated seed maturity of cotton. FIG. 1A shows 35-day-old cotton plants treated with water or acetic acid (20 mM supplemented to soil) or ethanol (50 mM supplemented to soil) for 2 days that were exposed to drought for 7 days, and then rewatered for 5 days. FIG. 1B shows starch staining of cotton leaves under well-watered and 6 days of drought treatment as described in FIG. 1A. FIG. 1C shows results related to enhanced photosynthesis performance of cotton after 6 days of drought treatment. FIG. 1D shows seed maturity of chemical-treated and water-treated cotton under well-watered conditions.

FIGS. 2A-2B provide experimental results demonstrating that acetic acid and ethanol treatment enhanced abiotic stress tolerance of sorghum. FIG. 2A shows 14-day-old sorghum plants treated with water or acetic acid (20 mM supplemented to soil) for 2 days that were exposed to drought for 14 days, and then rewatered for 3 days. FIG. 2B shows sorghum seeds treated with water or acetic acid (20 mM) or ethanol (50 mM) or combination of acetic acid (10 mM) and ethanol (25 mM) for 1 day were kept at 4° C. for 3 weeks, and then grown under well-watered conditions for 14 days. Plants were then exposed to drought and cold (4° C.) combination stress for 14 days, and then recovered for 2 days.

FIGS. 3A-3B provide experimental results demonstrating that acetic acid and ethanol treatment enhanced tolerance of corn to drought and heat combination stress. FIG. 3A shows 14-day-old corn plants treated with water or acetic acid (20 mM) or ethanol (50 mM) or combination of acetic acid (10 mM) and ethanol (25 mM) for 2 days that were exposed to drought and heat (40° C.) combination stress for 16 days, and then recovered for 2 days. FIG. 3B shows representative root of corn plants described in FIG. 3A.

FIGS. 4A-4C provide experimental results demonstrating that acetic acid and ethanol treatments enhanced drought tolerance of common bean. FIG. 4A shows 14-day-old common bean plants treated with water or acetic acid (20 mM supplemented to soil) or ethanol (50 mM supplemented to soil) for 2 days that were exposed to drought for 11 days, and then rewatered for 5 days. FIG. 4B shows the photosynthesis performance of acetic water-, acetic acid- and ethanol-treated plants exposed to drought for 5 days. FIG. 4C shows seed yield per plants of acetic water-, acetic acid- and ethanol-treated plants under drought and well-watered conditions.

FIGS. 5A-5I provide experimental results demonstrating that acetic acid treatment enhanced drought tolerance of common bean. FIG. 5A shows the phenotype of 2-week-old seedlings pretreated with 20 mM acetic acid or water-treated for 2 days followed by 9 days of water withdrawn. FIG. 5B shows the phenotype of seedlings after 11 days of drought treatments and 5 days of rewatering. FIG. 5C shows survival rate under drought in the presence or absence of acetic acid described in FIG. 5B. FIG. 5D shows relative leaf temperature of common bean plants pretreated with 20 mM acetic acid or water for 2 days, followed by drought treatment for 4 days. FIGS. 5E-5F show relative water contents (FIG. 5E) and electrolyte leakage rate (FIG. 5F) of acetic acid-pretreated and water-treated plants on 4^{th} and 5^{th} days after stress (das). FIGS. 5G-5I show Anthocyanin (FIG. 5G), total chlorophyll (FIG. 5H), and carotenoid (FIG. 5I) contents of common bean plants that were treated with acetic acid or water under drought stress.

FIGS. 6A-6F provide experimental results demonstrating that acetic acid treatment enhanced antioxidant capacity of common bean in response to drought stress. FIGS. 6A-6B show hydrogen peroxide (FIG. 6A) and malondialdehyde (MDA) (FIG. 6B) content of water- or acetic acid-treated plants exposed to drought stress treatment. FIGS. 6C-6F show superoxide dismutase (SOD) (FIG. 6C), ascorbate peroxidase (APX) (FIG. 6D), glutathione peroxidase (GPX) (FIG. 6E) and glutathione S-transferase (GST) (FIG. 6F) of water- and acetic acid-treated plants exposed to drought stress treatment.

FIGS. 7A-7G provide experimental results demonstrating that acetic acid treatments enhanced heat tolerance of common bean. FIG. 7A shows 14-day-old common bean plants treated with water or acetic acid (20 mM supplemented to soil) for 2 days that were exposed to heat (40° C.) stress for 18 days, and then rewatered for 5 days. FIG. 7B shows the survival rate of water- or acetic acid-treated plants after heat recovery. FIGS. 7C-7D show an electrolyte leakage rate (FIG. 7C), and total chlorophyll content (FIG. 7D) of water or acetic acid-treated plants exposed to heat stress treatment. FIG. 7E shows starch staining assay of water- or acetic acid-treated plants exposed to heat stress for 12 days. FIGS. 7F-7G show the photosynthesis performance of water- and acetic acid-treated plants exposed to heat stress for 12 days.

FIGS. 8A-8H provide experimental results demonstrating that acetic acid treatments enhanced antioxidant capacity of common bean in response to heat stress. FIGS. 8A-8D show anthocyanin (FIG. 8A), carotenoid (FIG. 8B), hydrogen peroxide (FIG. 8C), and malondialdehyde (MDA) (FIG. 8D) content of water- or acetic acid-treated plants exposed to heat stress treatment. FIGS. 8E-8H show superoxide dismutase (SOD) (FIG. 8E), ascorbate peroxidase (APX) (FIG. 8F), glutathione peroxidase (GPX) (FIG. 8G) and glutathione S-transferase (GST) (FIG. 8H) of water- and acetic acid-treated plants exposed to heat stress treatment.

FIGS. 9A-9D provide experimental results demonstrating transgenerational memory effects of acetic acid treatment on drought tolerance of soybean. FIG. 9A shows 16-day-old W1 and E1 soybean plants that were exposed to drought for 7 days. FIG. 9B shows 8-week-old W1 and E1 soybean plants that were exposed to drought for 10 days. FIGS. 9C-9D show hydrogen peroxide (FIG. 9C) and superoxide (FIG. 9D) accumulation of W1 and E1 leaf after 5 days of drought treatment as described in FIG. 9A.

FIGS. 10A-10B provide experimental results demonstrating the transgenerational memory effects of acetic acid and ethanol treatment on drought tolerance of common bean. FIG. 10A shows 16-day-old W1 and E1 common bean plants that were exposed to drought for 5 days. FIG. 10B shows the photosynthesis performance of W1 and E1 (ethanol) plants that were exposed to drought for 5 days as described in FIG. 10A.

FIGS. 11A-11C provide experimental results demonstrating that acetic acid and ethanol stimulated primary root growth in sorghum. FIGS. 11A-11B show root photos (FIG. 11A) and the primary root length (FIG. 11B) of three-day-old sorghum plants germinated from seeds primed with acetic acid (20 mM) or ethanol (50 mM) or water (control) for 16 hours and grown under normal conditions for 3 days. Error bars represent the means±standard errors (SEs) (n=20). Statistical significance was determined by a Student's t-test (***P<0.001). FIG. 11C shows confocal images of root tip of 3-day-old sorghum plants as described in FIG. 11A.

FIGS. 12A-12F provide experimental results demonstrating that acetic acid and ethanol stimulated shoot growth, inflorescence development and blooming in sorghum. FIG. 12A shows 49-day-old sorghum plants germinated from seeds primed with acetic acid (20 mM) or ethanol (50 mM) or water (control) for 16 hours that were grown under normal condition in the greenhouse. FIG. 12B shows leaf temperature of plants as described in FIG. 12A. FIGS. 12C-12D show inflorescence of 42-day-old and (FIG. 12C) and 55-day-old (FIG. 12D) sorghum plants as described in FIG. 12A. FIG. 12E shows the rate of flowering plants as described in FIG. 12A. Error bars represent the means±standard errors (n≥18). FIG. 12F shows 75-day-old plants germinated from seeds primed with acetic acid (20 mM) or ethanol (50 mM) or water (control) for 16 hours were grown under normal irrigation conditions in the field.

FIGS. 13A-13F provide experimental results demonstrating acetic acid- and ethanol-mediated transgenerational memory effects on stimulating plant growth and development of the second-generation sorghum. FIGS. 13A-13D provide root photos (FIG. 13A) and confocal microscopic images (FIGS. 13B-13D) of root tip of 3-day-old sorghum plants germinated from seeds primed with acetic acid (20 mM) or ethanol (50 mM) or water (control) for 16 hours and grown under normal conditions for 3 days. FIGS. 13E-13F show inflorescence of 35-day-old (FIG. 13E) and 49-day-old (FIG. 13F) sorghum plants germinated from seeds primed with acetic acid (20 mM) or ethanol (50 mM) or water (control) for 16 hours that were grown under normal irrigation condition in the greenhouse. EZ, elongation zone; TZ, transition zone; CDZ, cell division zone.

FIGS. 14A-14K provide experimental results demonstrating acetic acid and ethanol enhanced drought tolerance in sorghum. FIGS. 14A-14B show 21-day-old sorghum plants germinated from seeds primed with acetic acid (20 mM) or ethanol (50 mM) or water (control) for 16 hours that were exposed to drought for 7 days (FIG. 14A) and then rewatered for 4 days (FIG. 14B). FIGS. 14C-14I show leaf temperature (FIG. 14C), relative leaf water content (FIG. 14D), leaf water potential (FIG. 14E), electrolyte leakage (FIG. 14F), total chlorophyll (FIG. 14G), superoxide (FIG. 14H), and hydrogen peroxide accumulation (FIG. 14I) of acetic acid- or ethanol- or water-seed-primed plants at 6 days of drought treatment. Error bars represent the means±standard errors (SEs) (n=5). FIGS. 14J-14K provide representative photos of 10 seeds (FIG. 14J) and weight of 100 seeds (FIG. 14K) collected from acetic acid- or ethanol- or water-seed-primed plants grown under normal irrigation conditions in the greenhouse. Error bars represent the means±SEs (n=10). Statistical significance was determined by a Student's t-test (*P<0.05; **P<0.01; ***P<0.001).

FIGS. 15A-15F provide experimental results demonstrating acetic acid and ethanol enhanced combined drought and heat stress tolerance in sorghum. FIG. 15A shows an image of 21-day-old sorghum plants germinated from seeds primed with acetic acid (20 mM) or ethanol (50 mM) or water (control) for 16 hours that were exposed to combined drought and heat stress for 5 days. FIGS. 15B-15E show leaf temperature (FIG. 15B), relative water content (FIG. 15C), leaf water potential (FIG. 15D), and electrolyte leakage (FIG. 15E) of acetic acid- or ethanol- or water-seed-primed plants at 5 days of combined drought and heat stress treatment. Error bars represent the means±SEs (n=6). Statistical significance was determined by a Student's t-test (***P<0.001). FIG. 15F shows roots of acetic acid- or ethanol- or water-seed-primed plants followed by combined drought and heat stress treatment for 7 days.

FIGS. 16A-16K provide experimental results demonstrating acetic acid- and ethanol-mediated transgenerational memory effects on drought tolerance of the second-generation sorghum. FIGS. 16A-16B show images of 21-day-old second-generation sorghum plants that were exposed to drought for 7 days (FIG. 16A), and then rewatered for 5 days (FIG. 16B). FIGS. 16C-16E show leaf temperature (FIG. 16C), relative leaf water content (FIG. 16D), leaf water potential (FIG. 16E), electrolyte leakage (FIG. 16F), shoot and root dried weight (FIG. 16G), carbon assimilation rate (FIG. 16H), superoxide (FIG. 16I), and hydrogen peroxide (FIG. 16J) accumulation of the second-generation sorghum plants at 6 days of drought treatment, and greater seed weight per plants (FIG. 16K) than W1 plants. Error bars represent the means±SEs (n=5). Statistical significance was determined by a Student's t-test (*P<0.05; **P<0.01; ***P<0.001).

FIGS. 17A-17E provide experimental results demonstrating acetic acid- and ethanol-mediated transgenerational memory effects combined drought and heat tolerance of the second-generation sorghum. FIG. 17A shows images of 21-day-old second-generation sorghum plants that were exposed to combined drought and heat stress for 7 days followed by recovery for 5 days. FIGS. 17B-17E shows leaf temperature (FIG. 17B), relative water content (FIG. 17C), leaf water potential (FIG. 17D), and electrolyte leakage (FIG. 17E) of the second-generation sorghum at 6 days of combined drought and heat stress treatment. Error bars represent the means±SEs (n=6). Statistical significance was determined by a Student's t-test (***P<0.001).

FIGS. 18A-18G provide experimental results demonstrating that acetic acid and ethanol enhanced drought tolerance in cotton. FIG. 18A shows that 35-day-old cotton plants treated with 2 litters of acetic acid (20 mM) or ethanol (50 mM) or water (control) for 2 days were exposed to drought for 7 days and then rewatered for 5 days. FIGS. 18B and 18C show carbon assimilation (FIG. 18B) and water-use-efficiency (WUEᵢₙₜ) (FIG. 18C) of cotton acetic acid- or ethanol- or water-treated plants at 6 days of drought treatment. Error bars represent the means±standard errors (SEs) (n=3). FIG. 18D shows starch accumulation of cotton acetic acid- or ethanol- or water-treated plants at 6 days of drought treatment. FIGS. 18E-18G show relative leaf water content (FIG. 18E), electrolyte leakage (FIG. 18F), and total chlorophyll (FIG. 18G) of acetic acid- or ethanol- or water-treated plants at 6 days of drought treatment. Error bars represent the means±SEs (n=5). Statistical significance was determined by a Student's t-test (*P<0.05; **P<0.01; ***P<0.001).

FIGS. 19A-19H provide experimental results demonstrating that acetic acid and ethanol enhanced drought tolerance in common bean. FIG. 19A shows the phenotype of 14-day-old plants pretreated with 20 mM acetic acid or 50 mM ethanol or water for 2 days followed by 11 days of water withdrawal and then 5 days of rewatering. FIG. 19B shows the survival rates of acetic acid- or ethanol- or water-treated plants as described in FIG. 19A. Rate of survived plants (number of survived plants/total number of plants) was calculated using plants from 5 pots (10 plants/pot) at 5 days after rewatering. FIGS. 19C-19F show relative water contents (FIG. 19C), electrolyte leakage rates (FIG. 19D), total chlorophyll (FIG. 19E), and anthocyanin contents (FIG. 19F) of acetic acid- or ethanol- or water-treated plants on 5^{th} days after drought stress. Error bars represent the means±standard errors (SEs) (n=5). FIGS. 19G and 19H show carbon assimilation (FIG. 19G) and water-use-efficiency (WUEᵢₙₜ) (FIG. 19H) of acetic acid- or ethanol- or water-treated plants on 5^{th} days after drought stress. Error bars represent the means±SEs (n=3). Statistical significance was determined by a Student's t-test (*P<0.05; **P<0.01; ***P<0.001).

FIGS. 20A-20G provide experimental results demonstrating that acetic acid and ethanol enhanced heat tolerance in common bean. FIG. 20A shows the phenotype of 14-day-old plants pretreated with 20 mM acetic acid or 50 mM ethanol or water for 2 days followed by 18 days of heat stress treatment and then 5 days of rewatering. FIGS. 20B-20D show electrolyte leakage rates (FIG. 20B), total chlorophyll (FIG. 20C), and anthocyanin contents (FIG. 20D) of the acetic acid- or ethanol- or water-treated common bean plants on 14 days after heat stress. Error bars represent the means±standard errors (SEs) (n=5). FIGS. 20E-20G show carbon assimilation rate (FIG. 20E), starch (FIG. 20F) and hydrogen peroxide (FIG. 20G) accumulation of the acetic acid- or ethanol- or water-treated common bean plants on 12^{th} days after heat stress. Error bars represent the means±SEs (n=3). Statistical significance was determined by a Student's t-test (*P<0.05; **P<0.01; ***P<0.001).

FIGS. 21A-21E provide experimental results demonstrating that acetic acid and ethanol memorized its effects on enhancing drought tolerance in the second-generation cotton. FIGS. 21A and 21B show 21-day-old second-generation cotton plants were exposed to drought for 12 days (FIG. 21A), and then rewatered for 4 days (FIG. 21B). FIGS. 21B and 21C show rate of survival plants as described in (FIG. 21A). Error bars represent the means±standard errors (SEs) (n=36). FIG. 21D shows carbon assimilation rate of the second-generation cotton plants at 6 days of drought treatment. Error bars represent the means±SEs (n=3). Statistical significance was determined by a Student's t-test (*P<0.05; **P<0.01; ***P<0.001). FIG. 21E shows representative roots of the second-generation cotton plants as described in (FIG. 21A).

FIGS. 22A-22G provide experimental results demonstrating that acetic acid memorized its effects on enhancing drought tolerance in the second-generation cotton. FIGS. 22A-22D show starch accumulation (FIG. 22A), relative water content (FIG. 22B), electrolyte leakage (FIG. 22C), and superoxide and hydrogen peroxide accumulation of the second-generation cotton plants at 6 days of drought treatment (FIG. 22D). Error bars represent the means±SEs (n=5). FIGS. 22E-22G show total lipid (FIG. 22E), soluble protein (FIG. 22F), and total carbohydrate content (FIG. 22G) of W1 and A1 cotton seeds. Error bars represent the means±SEs (n=4). Statistical significance was determined by a Student's t-test (*P<0.05; **P<0.01; ***P<0.001). DAB, 3,3-diaminobenzidine; das, days after stress; DW, dried weight; NBT, nitro blue tetrazolium.

FIGS. 23A-23B provide experimental results demonstrating that acetic acid and ethanol memorized their effects on enhancing drought and heat tolerance in the second-generation common bean. FIG. 23A shows 14-day-old second-generation common bean plants were exposed to drought for 11 days, and then rewatered for 4 days. FIG. 23B shows 14-day-old second-generation common bean plants were exposed to heat stress for 14 days, and then rewatered for 4 days.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Climate change has escalated environmental stresses, resulting in adverse effects on plant growth, plant development, and crop productivity. Thus, the development of abiotic stress-tolerant crops is timely and necessary to minimize the negative effect of stress on productivity.

In the last decade, the role of epigenetics and their effects on gene expression in response to environmental stress has gained more attention. Abiotic stresses can lead to epigenetic modifications, which change chromatin structure and gene expression to play an important function in plant stress adaptation. Moreover, stable epigenetic modifications could be inherited in a generation and across generations, which could help plants adapt to abiotic stress conditions. These findings suggest that epigenomes could be a target for developing climate-resistant crops.

Recent studies have revealed that various chemical compounds could help plants improve abiotic stress tolerances. Various chemicals have potential applications for agricultural biotechnology and have helped decrease the cultivation and management cost.

As such, chemical application could be one of the most promising methods for enhancing abiotic stress tolerance of plants and seeds in growth fields. However, because of the negative environmental impacts, such as the inhibition of plant growth, and the reduced effectiveness after prolonged application, safer and more effective alternative chemicals are required.

In addition, to address the challenges and opportunities of chemical application in crop stress management, the molecular events underlying the chemicals-controlled adaptive mechanisms that regulate plant environmental stress responses need to be determined. Numerous embodiments of the present disclosure address the aforementioned needs.

### Methods of Enhancing Plants and Seeds

In some embodiments, the present disclosure pertains to methods of enhancing a physiological performance or environmental stress resistance of a plant or seed. In some embodiments, the methods of the present disclosure include a step of exposing the plant or seed to a composition. In some embodiments, the composition includes one or more active ingredients. In some embodiments, the active ingredients include, without limitation, ethanol, acetic acid, and combinations thereof. As set forth in more detail herein, the methods of the present disclosure can have numerous embodiments.

### Exposure of Plants and Seeds to Compositions

Various methods may be utilized to expose the plants and seeds of the present disclosure to a composition. For instance, in some embodiments, the exposing includes spraying the plant or seed with the composition. In some embodiments, the exposing includes soaking the plant or seed with the composition. In some embodiments, the exposing includes pouring the composition onto the plant or seed. In some embodiments, the exposing occurs through the utilization of water baths, irrigation, or combinations thereof.

Plants and seeds may be exposed to a composition of the present disclosure for various periods of time. For instance, in some embodiments, the exposure occurs for at least one day. In some embodiments, the exposure occurs for at least two days. For instance, in some embodiments, plants and seeds (e.g., cotton or common bean plants) may be treated with 2 litters (L) of acetic acid or ethanol or water that is added to a tray containing 18 pots (i.e., 0.7 L per pot) for two days.

### Enhancement of Physiological Performance

The exposure of plants and seeds of the present disclosure to a composition of the present disclosure can have various effects on the plant or seed. For instance, in some embodiments, the exposure enhances the physiological performance of the plant or seed relative to an untreated plant or seed, respectively. In some embodiments, the enhanced physiological performance includes, without limitation, reduced water consumption, enhanced photosynthetic performance, an increase in number of photosynthetic pigments (e.g., chlorophyll, carotenoid and/or anthocyanin), enhanced antioxidant defense, enhanced antioxidant accumulation, enhanced flowering, enhanced seed maturity, enhanced growth, an increase in soluble proteins, an increase in starch, increased seed yield, a reduction in water loss, reduced electrolyte leakage, a reduction in reactive oxygen species (ROS) accumulation, lower malondialdehyde accumulation, enhanced root growth, enhanced shoot growth, reduced leaf temperatures, and combinations thereof.

In some embodiments, the enhanced physiological performance includes enhanced protein accumulation in the plant or seed. In some embodiments, the accumulated protein includes, without limitation, superoxide dismutase (SOD), ascorbate peroxidase (APX), glutathione peroxidase (GPX), glutathione S-transferase (GST) accumulation, and combinations thereof.

In some embodiments, the enhanced physiological performance is inheritable in the plant or seed. For instance, in some embodiments, the offspring plants or seeds from the treated plant or seed demonstrate substantially the same enhanced physiological performance as the treated plant or seed, respectively. In some embodiments, the offspring plants or seeds from the treated plant or seed demonstrate at least about 70% of the same enhanced physiological performance as the treated plant or seed, respectively. In some embodiments, the offspring plants or seeds from the treated plant or seed demonstrate at least about 80% of the same enhanced physiological performance as the treated plant or seed, respectively.

Without being bound by theory, enhanced physiological performance can occur through various mechanisms. In some embodiments, the enhanced physiological performance occurs through inheritable epigenetic modifications. In some embodiments, the epigenetic modifications include, without limitation, DNA methylation, histone modification, small interfering RNA (siRNA) modification, increased gene expression, and combinations thereof. For instance, as described in the Examples, expression of several genes involved in DNA methylation in Arabidopsis was changed in ethanol-treated plants compared with water-treated plants.

### Enhancement of Resistance to Environmental Stress

In some embodiments, the exposure of plants and seeds of the present disclosure to a composition of the present disclosure enhances the resistance of the plant or seed to one or more environmental stresses relative to an untreated plant or seed, respectively.

In some embodiments, the one or more environmental stresses include, without limitation, drought, heat, freezing temperatures, microbial contamination, biotic stress, abiotic stress, plant pathogenesis, and combinations thereof. In some embodiments, the one or more environmental stresses include abiotic stress. In some embodiments, the one or more environmental stresses include drought.

In some embodiments, the enhanced environmental stress resistance is inheritable in the plant or seed. For instance, in some embodiments, the offspring plants or seeds from the treated plants or seeds demonstrate substantially the same resistance to the one or more environmental stresses as the treated plant or seed, respectively. In some embodiments, the offspring plants or seeds from the treated plants or seeds demonstrate at least about 70% of the same resistance to the one or more environmental stresses as the treated plant or seed, respectively. In some embodiments, the offspring plants or seeds from the treated plants or seeds demonstrate at least about 80% of the same resistance to the one or more environmental stresses as the treated plant or seed, respectively.

Without being bound by theory, enhanced environmental stress resistance can occur through various mechanisms. In some embodiments, the enhanced environmental stress resistance occurs through inheritable epigenetic modifications. In some embodiments, the epigenetic modifications include, without limitation, DNA methylation, histone modification, small interfering RNA (siRNA) modification, increased gene expression, and combinations thereof.

### Plants and Seeds

The methods of the present disclosure may be utilized to enhance the physiological performance or environmental stress resistance of various plants and seeds. For instance, in some embodiments, the treated plant or seed includes, without limitation, maize, rice, bean, soybean, common bean, pinto bean, corn, cotton, wheat, N. benthamiana, Arabidopsis, tobacco, tomato, lettuce, potato, grapes, sorghum, varieties thereof, and combinations thereof. In some embodiments, the treated plant or seed includes, without limitation, soybean, common bean, pinto bean, corn, cotton, Arabidopsis, sorghum, varieties thereof, and combinations thereof. In some embodiments, the treated plant or seed includes sorghum.

In some embodiments, the treated plant or seed includes a treated plant. In some embodiments, the treated plant demonstrates enhanced physiological performance relative to an untreated plant. In some embodiments, the treated plant demonstrates enhanced resistance to one or more environmental stresses relative to an untreated plant.

In some embodiments, the compositions of the present disclosure may be exposed to a plant by applying the composition to the soil of the plant. For instance, in some embodiments, 2 litters (L) of acetic acid or ethanol or water may be added to the soil of the plant (e.g., cotton or common bean plants). In some embodiments, the methods of the present disclosure also include a step of collecting offspring seeds from a treated plant and growing offspring plants from the offspring seeds.

In some embodiments, the enhanced environmental stress resistance is inheritable in the offspring plants. In some embodiments, the offspring plants demonstrate substantially the same resistance to one or more environmental stresses as the treated plant.

In some embodiments, the enhanced physiological performance is inheritable in the offspring plants. In some embodiments, offspring plants demonstrate substantially the same enhanced physiological performance as the treated plant.

In some embodiments, the treated plant or seed includes a treated seed. In some embodiments, the treated seed demonstrates enhanced physiological performance relative to an untreated seed. In some embodiments, the treated seed demonstrates enhanced resistance to one or more environmental stresses relative to an untreated seed.

In some embodiments, the compositions of the present disclosure may be exposed to a seed by soaking the seed in the composition. In some embodiments, the soaking occurs for at least 12 hours. In some embodiments, the soaking occurs for at least 16 hours. For instance, in some embodiments, sorghum or cotton seeds may be treated with acetic acid or ethanol or water for 16 hours.

In some embodiments, the methods of the present disclosure also include a step of germinating the treated seeds to produce offspring plants from the treated seeds. In some embodiments, the offspring plants demonstrate enhanced physiological performance relative to an untreated plant. In some embodiments, the offspring plants demonstrate enhanced resistance to one or more environmental stresses relative to an untreated plant.

### Compositions

Plants and seeds may be exposed to various compositions. Such compositions generally include one or more active ingredients that include ethanol, acetic acid, or combinations thereof. Additional embodiments of the present disclosure pertain to such compositions. As set forth in more detail herein, the compositions of the present disclosure can include numerous variations.

In some embodiments, the one or more active ingredients in the compositions of the present disclosure include ethanol. In some embodiments, the concentration of the ethanol in the composition is from about 5 mM to about 100 mM. In some embodiments, the concentration of the ethanol in the composition is at least about 10 mM. In some embodiments, the concentration of the ethanol in the composition is at least about 20 mM. In some embodiments, the concentration of the ethanol in the composition is at least about 25 mM. In some embodiments, the concentration of the ethanol in the composition is at least about 30 mM. In some embodiments, the concentration of the ethanol in the composition is at least about 40 mM. In some embodiments, the concentration of the ethanol in the composition is at least about 50 mM. In some embodiments, plants (e.g., sorghum or cotton or common bean or corn) may be treated with 50 mM ethanol or combined two chemicals (e.g., 10 mM acetic acid and 25 mM ethanol) for 16 hours.

In some embodiments, the one or more active ingredients in the compositions of the present disclosure include acetic acid. In some embodiments, the concentration of the acetic acid in the composition is between about 0.5 mM to about 30 mM. In some embodiments, the concentration of the acetic acid in the composition is at least about 1 mM. In some embodiments, the concentration of the acetic acid in the composition is at least about 5 mM. In some embodiments, the concentration of the acetic acid in the composition is at least about 10 mM. In some embodiments, the concentration of the acetic acid in the composition is at least about 20 mM. In some embodiments, plants (e.g., sorghum or cotton or common bean or corn) may be treated with 20 mM acetic acid or combined two chemicals (e.g., 10 mM acetic acid and 25 mM ethanol) for 16 hours.

In some embodiments, the one or more active ingredients in the compositions of the present disclosure include ethanol and acetic acid. In some embodiments, the concentration of the ethanol in the composition is at least about 25 mM and the concentration of the acetic acid in the composition is at least about 10 mM. In some embodiments, the concentration of the ethanol in the composition is at least about 50 mM, and the concentration of the acetic acid in the composition is at least about 20 mM. In some embodiments, plants (e.g., sorghum or cotton or common bean or corn) may be treated with 20 mM acetic acid or 50 mM ethanol or combined two chemicals (10 mM acetic acid and 25 mM ethanol) for 16 hours.

### Modified Plants and Seeds

Additional embodiments of the present disclosure pertain to modified plants or seeds that demonstrate enhanced physiological performance, enhanced environmental stress resistance, or combinations thereof. In some embodiments, the modified plants or seeds of the present disclosure are formed by the methods of the present disclosure. In some embodiments, the modified plants or seeds are formed by exposing the plants or seeds to a composition of the present disclosure (i.e., a composition that includes one or more active ingredients, where the active ingredients include ethanol, acetic acid, and combinations thereof).

In some embodiments, the modified plant or seed demonstrates enhanced physiological performance of the plant or seed relative to an untreated plant or seed, respectively. In some embodiments, the enhanced physiological performance includes, without limitation, reduced water consumption, enhanced photosynthetic performance, an increase in number of photosynthetic pigments (e.g., chlorophyll, carotenoid and anthocyanin), enhanced antioxidant defense, enhanced antioxidant accumulation, enhanced flowering, enhanced seed maturity, enhanced growth, an increase in soluble proteins, an increase in starch, increased seed yield, a reduction in water loss, reduced electrolyte leakage, a reduction in reactive oxygen species (ROS) accumulation, lower malondialdehyde accumulation, enhanced root growth, enhanced shoot growth, reduced leaf temperatures, and combinations thereof.

In some embodiments, the enhanced physiological performance includes enhanced protein accumulation in the plant or seed. In some embodiments, the accumulated protein includes, without limitation, superoxide dismutase (SOD), ascorbate peroxidase (APX), glutathione peroxidase (GPX), glutathione S-transferase (GST) accumulation, and combinations thereof.

In some embodiments, the enhanced physiological performance is inheritable in the plant or seed. In some embodiments, the offspring plants or seeds from the modified plant or seed demonstrate substantially the same enhanced physiological performance as the modified plant or seed, respectively. In some embodiments, the offspring plants or seeds from the modified plant or seed demonstrate at least about 70% of the same enhanced physiological performance as the modified plant or seed, respectively. In some embodiments, the offspring plants or seeds from the modified plant or seed demonstrate at least about 80% of the same enhanced physiological performance as the modified plant or seed, respectively.

In some embodiments, the enhanced physiological performance occurs through inheritable epigenetic modifications. In some embodiments, the epigenetic modifications include, without limitation, DNA methylation, histone modification, small interfering RNA (siRNA) modification, increased gene expression, and combinations thereof.

In some embodiments, the modified plant or seed demonstrates enhanced resistance of the plant or seed to one or more environmental stresses relative to an unmodified plant or seed, respectively. In some embodiments, the one or more environmental stresses include, without limitation, drought, heat, freezing temperatures, microbial contamination, biotic stress, abiotic stress, plant pathogenesis, and combinations thereof.

In some embodiments, the one or more environmental stresses include abiotic stress. In some embodiments, the one or more environmental stresses include drought. In some embodiments, the enhanced environmental stress resistance is inheritable in the plant or seed.

In some embodiments, the offspring plants or seeds from the modified plants or seeds demonstrate substantially the same resistance to the one or more environmental stresses as the modified plant or seed, respectively. In some embodiments, the offspring plants or seeds from the modified plants or seeds demonstrate at least about 70% of the same resistance to the one or more environmental stresses as the modified plant or seed, respectively. In some embodiments, the offspring plants or seeds from the modified plants or seeds demonstrate at least about 80% of the same resistance to the one or more environmental stresses as the modified plant or seed, respectively.

In some embodiments, the enhanced environmental stress resistance occurs through inheritable epigenetic modifications. In some embodiments, the epigenetic modifications include, without limitation, DNA methylation, histone modification, small interfering RNA (siRNA) modification, increased gene expression, and combinations thereof.

In some embodiments, the modified plant or seed includes, without limitation, maize, rice, bean, soybean, common bean, pinto bean, corn, cotton, wheat, N. benthamiana, Arabidopsis, tobacco, tomato, lettuce, potato, grapes, sorghum, varieties thereof, and combinations thereof. In some embodiments, the modified plant or seed includes, without limitation, soybean, common bean, pinto bean, corn, cotton, Arabidopsis, sorghum, varieties thereof, and combinations thereof. In some embodiments, the modified plant or seed includes sorghum.

In some embodiments, the modified plant or seed includes a modified plant. In some embodiments, the modified plant demonstrates enhanced physiological performance relative to an unmodified plant. In some embodiments, the modified plant demonstrates enhanced resistance to one or more environmental stresses relative to an unmodified plant. In some embodiments, the enhanced environmental stress resistance is inheritable in offspring plants from the modified plant. In some embodiments, the offspring plants demonstrate substantially the same resistance to one or more environmental stresses as the modified plant.

In some embodiments, the enhanced physiological performance is inheritable in offspring plants. In some embodiments, the offspring plants demonstrate substantially the same enhanced physiological performance as the modified plant.

In some embodiments, the modified plant or seed includes a modified seed. In some embodiments, the modified seed demonstrates enhanced physiological performance relative to an unmodified seed. In some embodiments, the modified seed demonstrates enhanced resistance to one or more environmental stresses relative to an unmodified seed.

In some embodiments, offspring plants from the modified seed demonstrate enhanced physiological performance relative to an unmodified plant. In some embodiments, offspring plants from the modified seed demonstrate enhanced resistance to one or more environmental stresses relative to an unmodified plant.

### Applications and Advantages

The methods, plants and seeds of the present disclosure provide numerous advantages. For instance, in some embodiments, the methods of the present disclosure produce plants and seeds that demonstrate enhanced physiological performance and environmental resistance in an inheritable manner, and without the need to utilize conventional chemical treatments. Rather, the methods of the present disclosure utilize safe, readily available and biodegradable chemicals. Moreover, the plants and seeds of the present disclosure demonstrate enhanced physiological performance and environmental resistance without being labeled as a genetically modified (GMO) crop.

As such, the methods, plants and seeds of the present disclosure can have numerous applications. For instance, in some embodiments, the methods, plants and seeds of the present disclosure can have agricultural applications, especially in regions that experience environmental stress.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Effects of Ethanol and Acetic Acid in Multiple Plant Species

In this Example, Applicant describes the development of a new memory stimulating system utilizing biological chemicals applied by various methods to activate genetic and epigenetic mechanisms in multiple plant species. Such methods enable the plants to inherit and take advantage of these advanced enhancements and/or positive effects on multiple plant metabolic pathways and stress tolerance across generations with a high degree of replication that is commercially acceptable and does not require extensive investments in breeding programs and trait expression programs (e.g., GMO technology).

Accordingly, the methods and systems described in this Example are appealing to multiple commercial plant-based operations supporting sustainability. For example, under drought stress conditions, in common bean, cotton, sorghum and soybean, the memory stimulating biological chemicals (MSBC) (ethanol and acetic acid) application helps plants reduce water consumption, enhance photosynthetic performance and antioxidant defense, stimulate flowering and seed maturity processes, and maintain plant growth and productivity.

Besides abiotic stress responses, data and examples of other positive effects has also been collected demonstrating influences of plant sustainability, including plant productivity, plant breeding and seed processing uniqueness and post harvesting longevity.

In proof-of-concept experiments, the first and second generation of MSBC-treated plants (Arabidopsis, soybean, pinto bean, sorghum, cotton) showed enhanced abiotic stress (drought) tolerance. The photosynthetic performance was greater in MSBC-treated plants as compared with water-treated (control) plants under drought and heat stress conditions. In addition, the MSBC-treated plants accumulated more antioxidants, photosynthetic pigments, soluble proteins and starch, and had greater antioxidant-enzyme activity than that in water-treated plants. Finally, the seed yield of MSBC-treated plants was higher than water-treated plants under abiotic stress (drought) conditions.

Applicant has identified that 54 potential MSBCs (including synthesized and natural plant-based chemicals) enhanced abiotic stress tolerance in plants. For example, Applicant found plant-based MSBC (ethanol and acetic acid) treatments enhanced drought tolerance of soybean, corn, cotton, and sorghum, and enhanced drought and heat tolerance in common bean in the first-generation. Importantly, Applicant found memory effect of plant-based MSBC (ethanol and acetic acid) treatments enhanced drought tolerance and maintained productivity in the second-generation cotton, sorghum, soybean and common bean.

These data suggest the existence of inherited memory effects of these plant-based MSBC treatment on plant sustainability and abiotic stress tolerance across generations in plants. Applicant's results provide the first view to the elucidation of molecular (genetic and epigenetic), morphological, biochemical and physiological mechanisms of MSBC involved in controlling plant sustainability and plant stress responses. Using plant-based MSBC treatment to activate the inherited epigenetic effects.

### Example 1.1. MSBC (Acetic Acid and Ethanol) Treatment Enhanced Drought Tolerance of Cotton

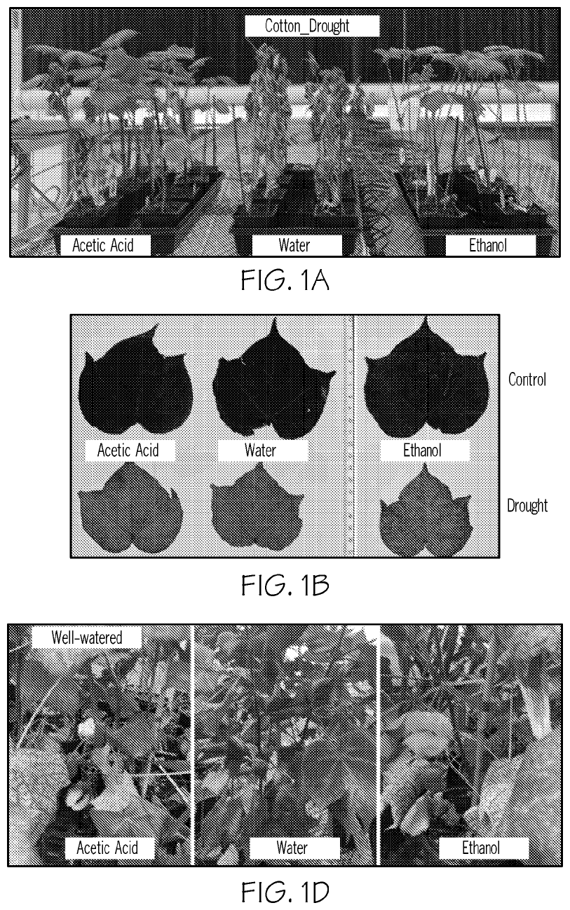

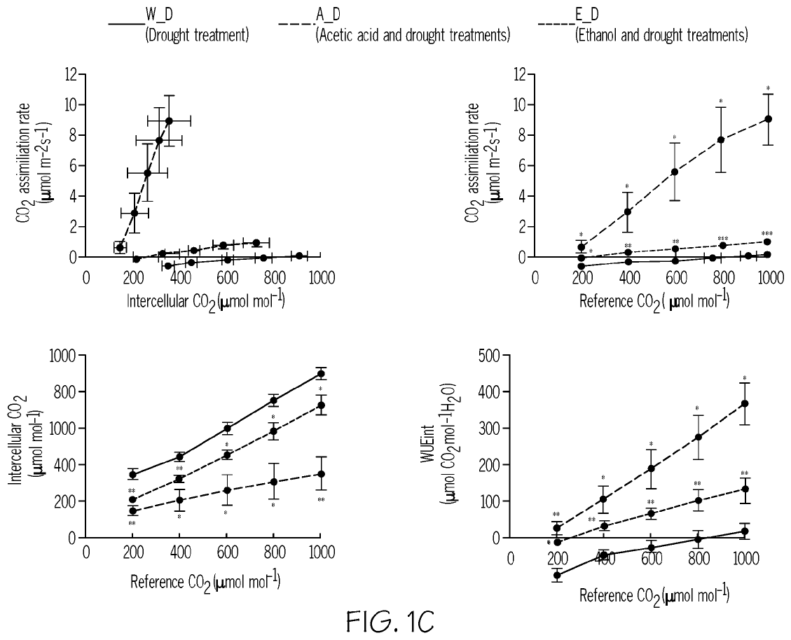

In this Example, Applicant showed that treatments of cotton with 20 mM acetic acid or 50 mM ethanol supplemented to soil improved drought tolerance (FIG. 1A). The MSBC-treated plants showed greater starch accumulation in leaf (FIG. 1B) and higher photosynthetic performance under drought stress conditions (FIG. 1C). In addition, the MSBC-treated plants showed earlier seed maturity under normal growth conditions (FIG. 1D).

### Example 1.2. MSBC (Acetic Acid and Ethanol) Treatment Enhanced Tolerance of Sorghum to Drought or Combination of Drought and Cold Stress

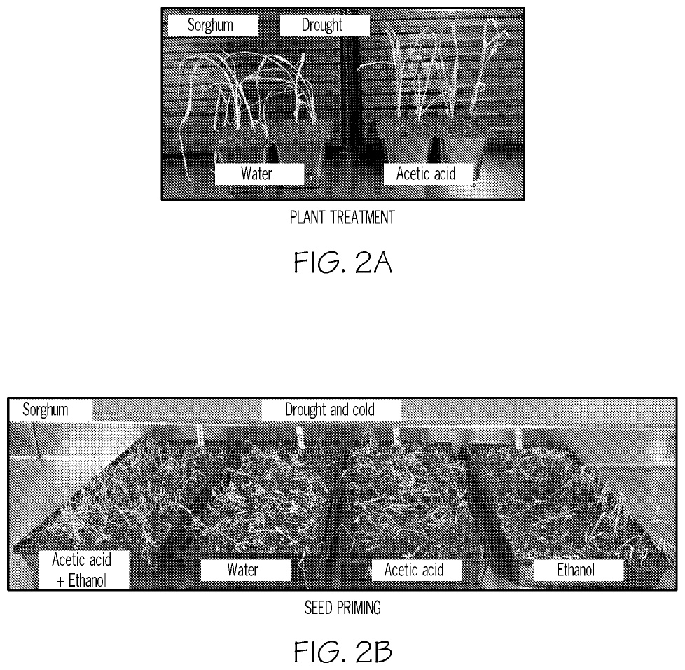

In this Example, Applicant showed that treatments of sorghum with 20 mM acetic acid supplemented to soil improved its drought tolerance (FIG. 2A). In addition, Applicant found that priming of sorghum seed with MSBCs resulted in enhanced tolerance to drought and cold combination stress (FIG. 2B).

### Example 1.3. MSBC (Acetic Acid and Ethanol) Treatment Enhanced Tolerance of Corn to Combination of Drought and Heat Stress

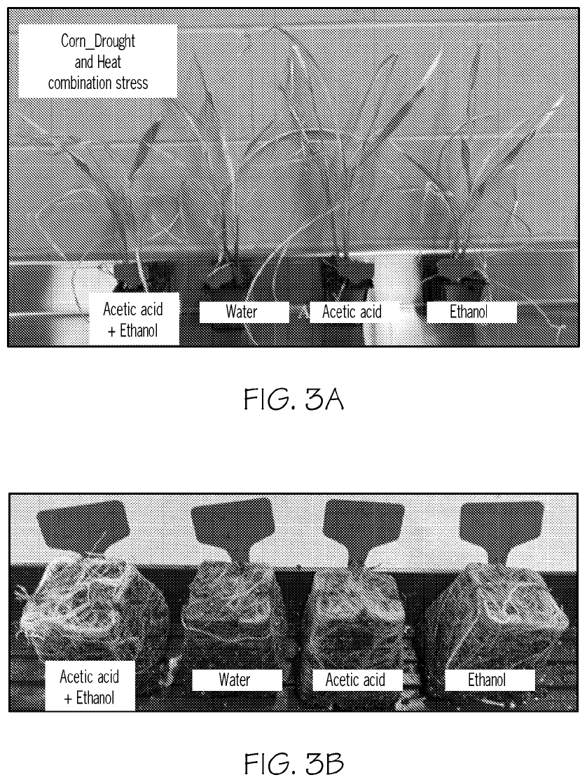

In this Example, Applicant showed that treatments of corn (maize) with acetic acid (20 mM) or ethanol (50 mM) or combination of acetic acid (10 mM) and ethanol (25 mM) supplemented to soil for 2 days improved its tolerance to drought and heat combination stress (FIGS. 3A-3B).

### Example 1.4. MSBC (Acetic Acid and Ethanol) Treatment Enhanced Drought and Heat Tolerance of Common Bean

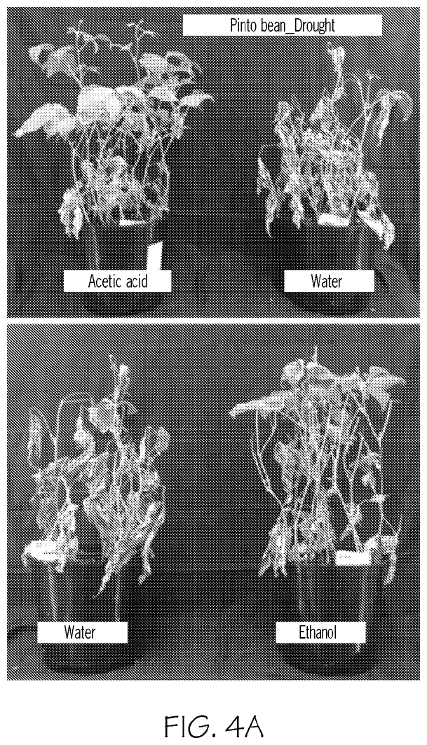

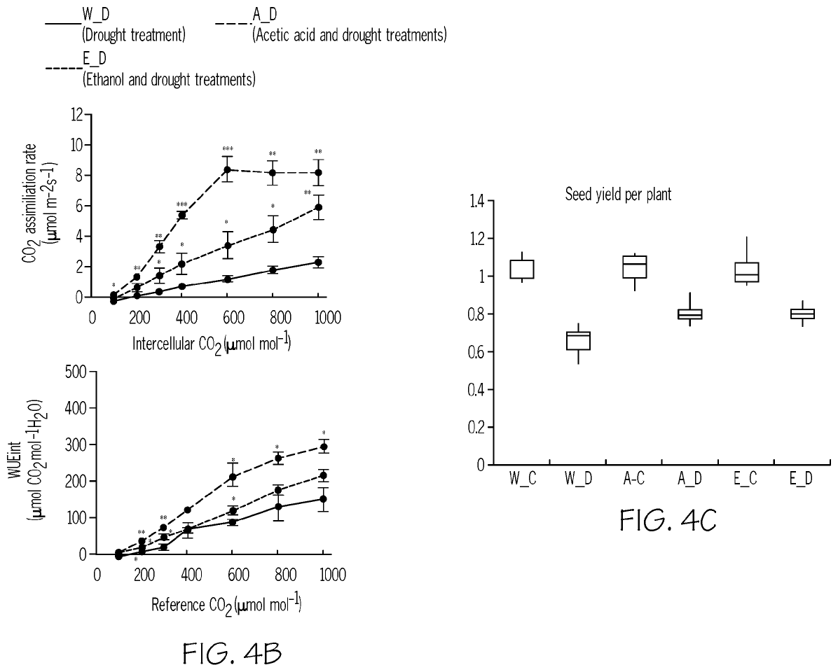

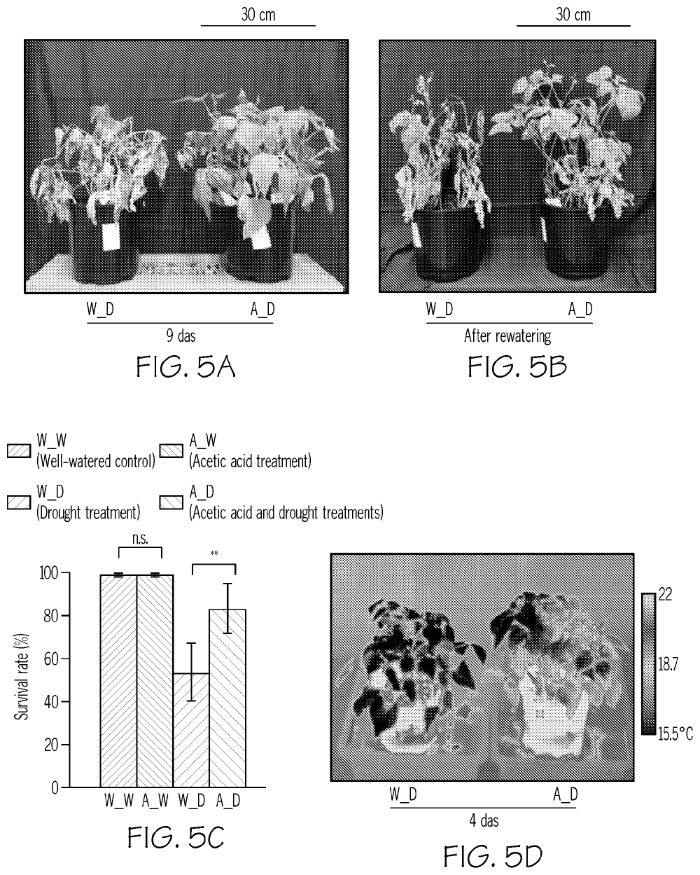

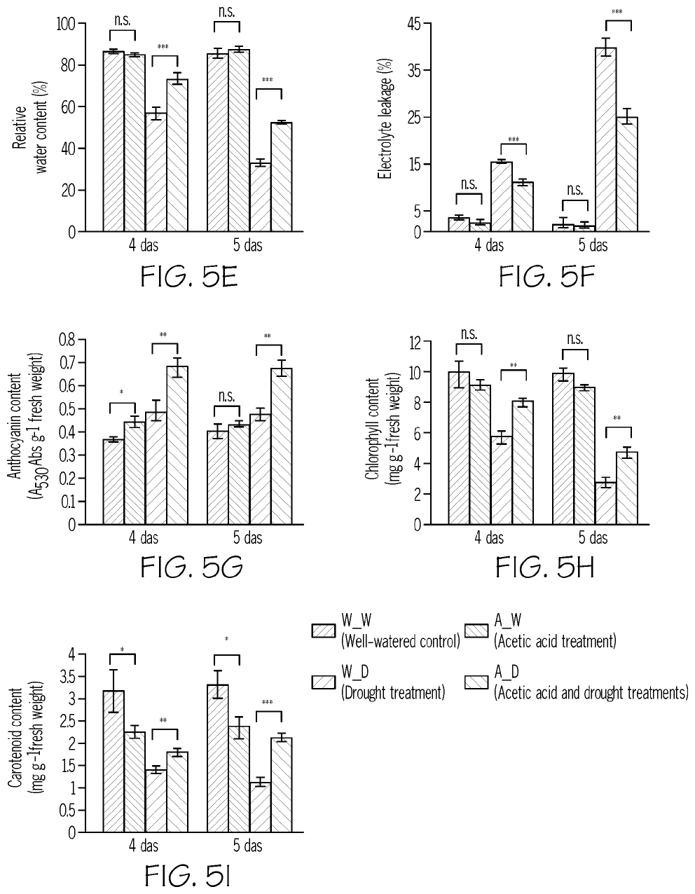

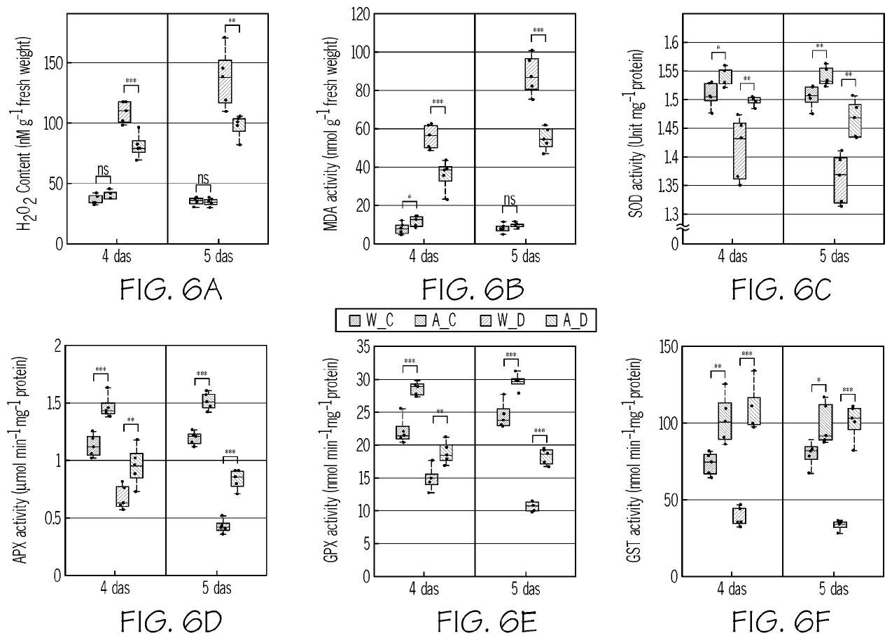

In this Example, Applicant has shown that treatments of common bean (pinto bean) with acetic acid (20 mM) or ethanol (50 mM) supplemented to soil for 2 days improved its drought and heat tolerance (FIGS. 4A-4C, 5A-5I, 6A-6F, 7A-7G, and 8A-8H).

The MSBC-treated plants showed higher photosynthetic performance and water use efficiency, higher seed yield per plant, higher survival plant rate, higher relative water content, lower electrolyte leakage rate, higher content of chlorophyll, carotenoid and anthocyanin, lower reactive oxygen species (ROS) and malondialdehyde accumulation and greater antioxidant enzyme activity under drought stress conditions (FIGS. 4A-4C, 5A-5I, 6A-6F).

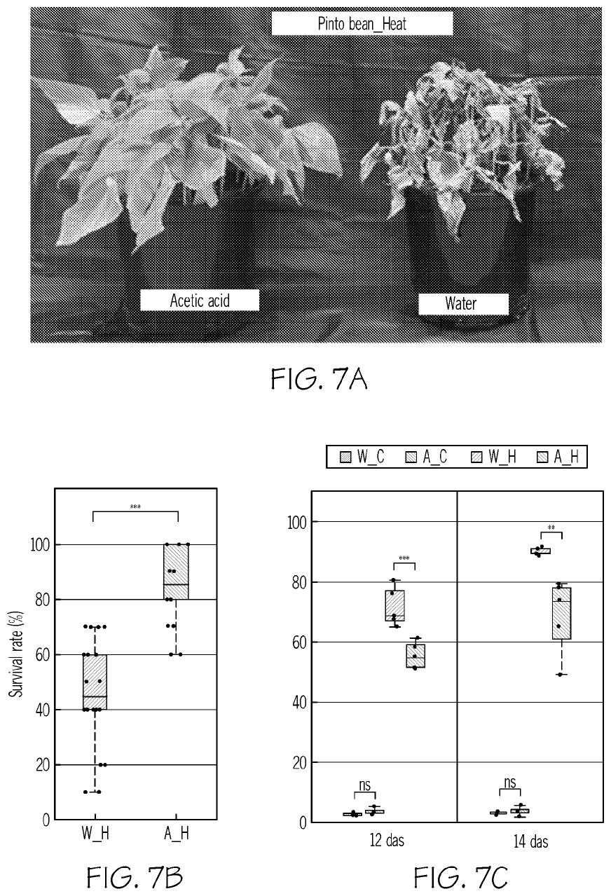

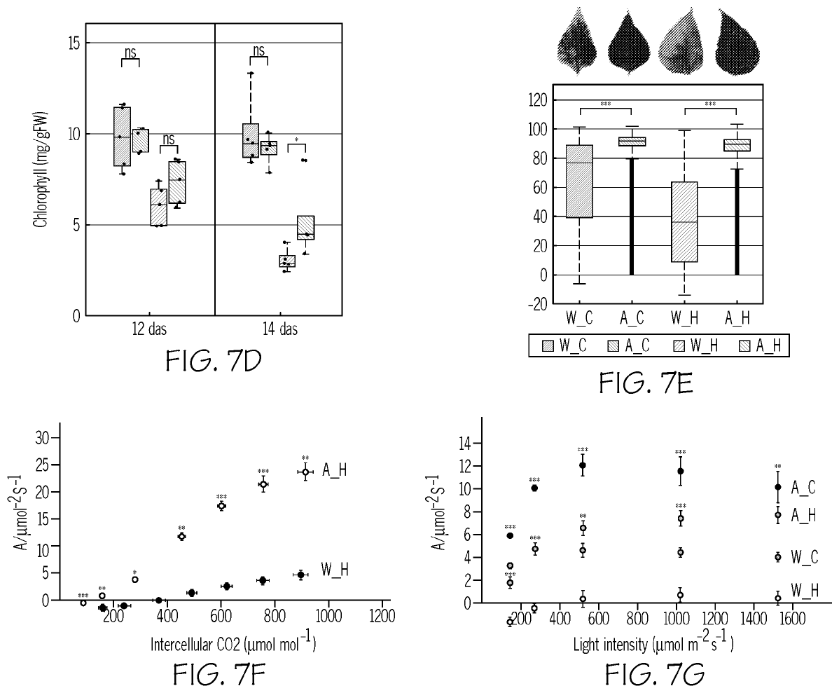

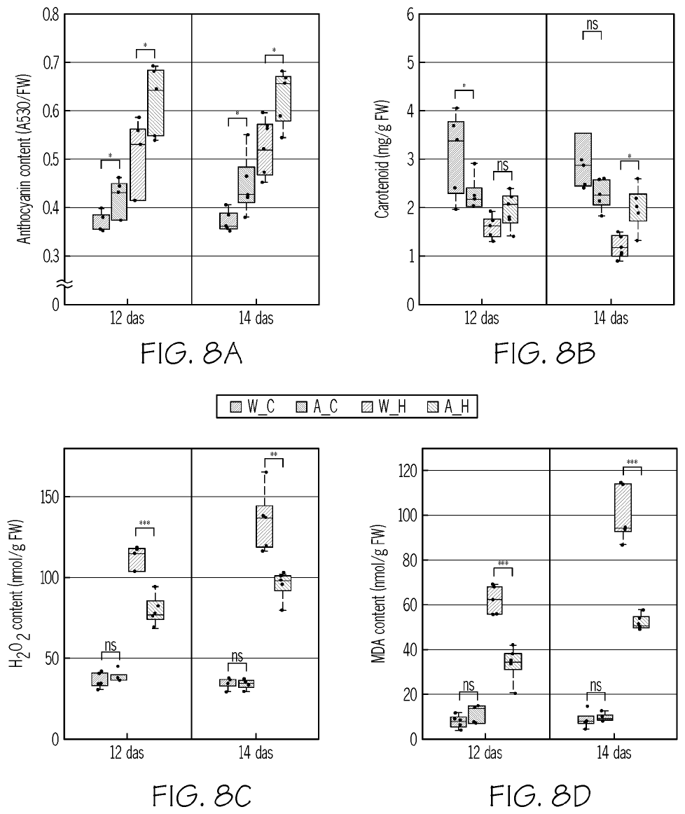

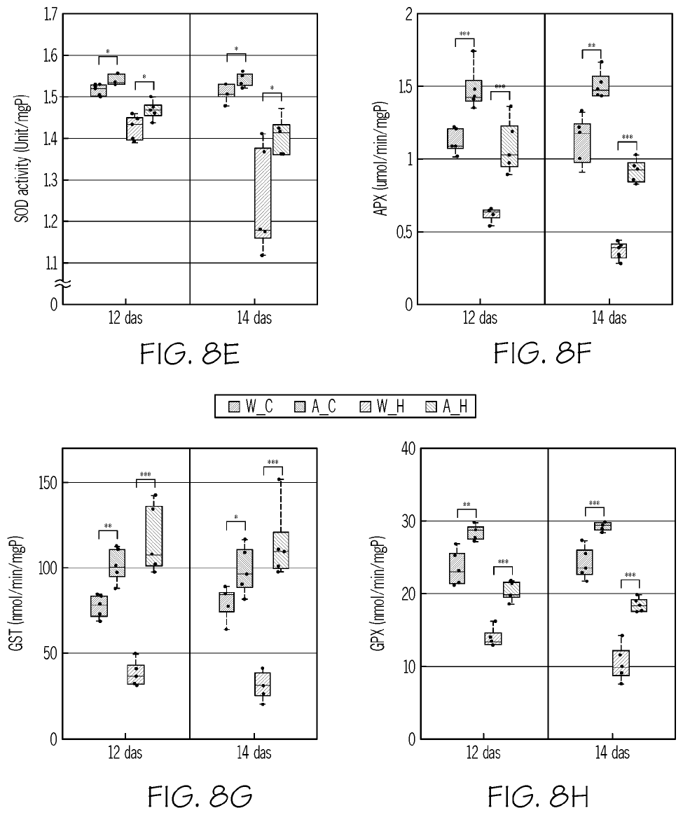

Applicant also observed that the MSBC-treated plants showed higher survival plant rate, higher photosynthetic performance, lower electrolyte leakage rate, higher content of chlorophyll, carotenoid and anthocyanin, greater starch accumulation, lower ROS and malondialdehyde accumulation, and greater antioxidant enzyme activity under heat stress conditions (FIGS. 7A-7G and 8A-8H).

### Example 1.5. MSBC (Acetic Acid and Ethanol) Mediated Transgenerational Memory Effects on Drought Tolerance of the Second-Generation Soybean and Common Bean

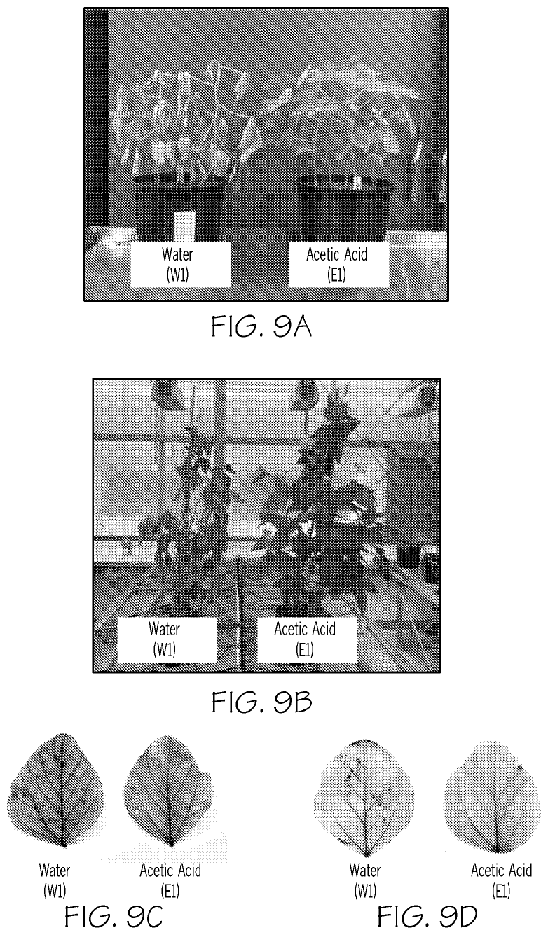

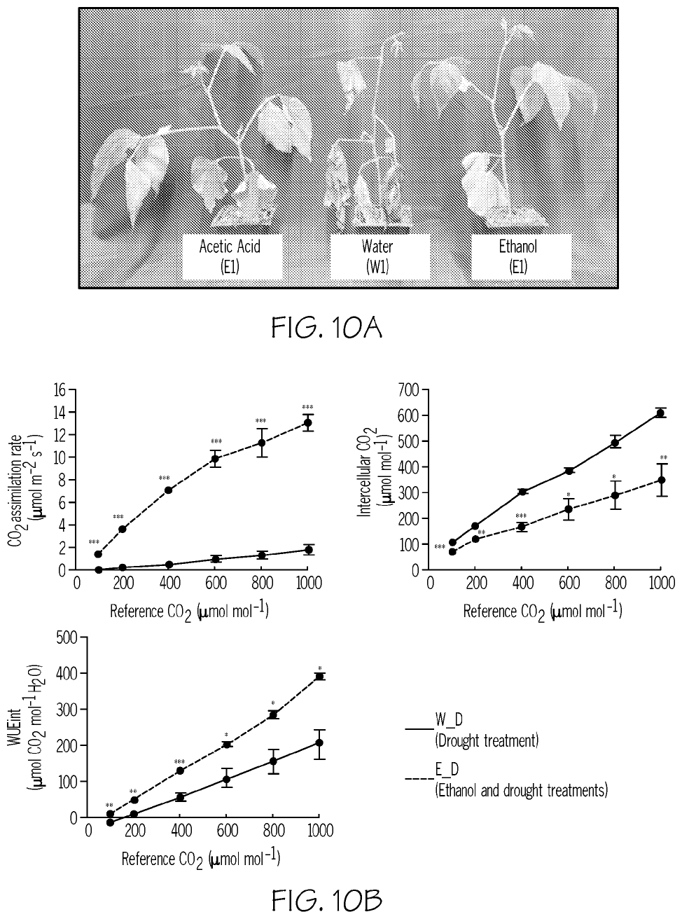

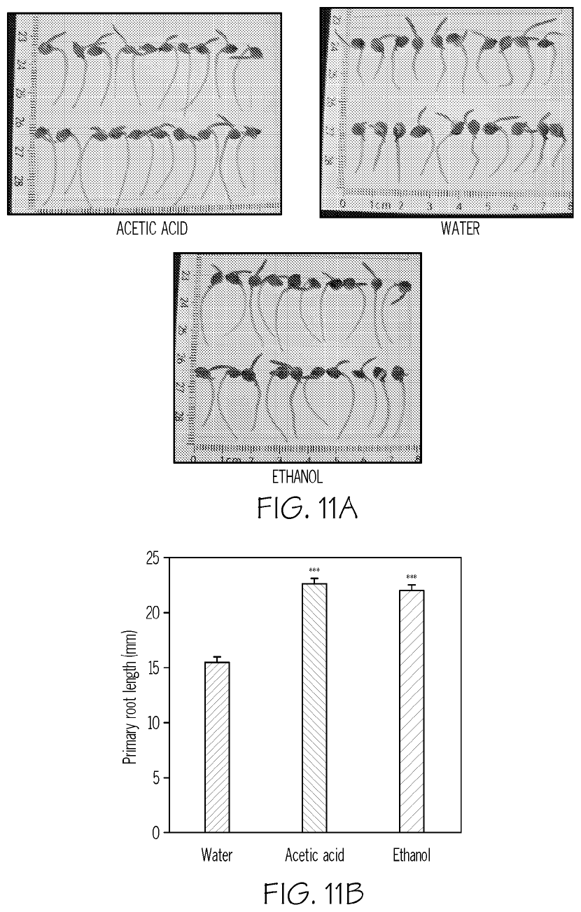

To identify whether MSBC treatment induces its memory effect leading to drought tolerance of second-generation soybean and common bean plants, 14-day-old soybean or common bean plants were treated with water (control) or MSBCs for 2 days, and then continued to grow under non-stressed conditions until harvest [seeds harvested from water-treated (W0) and MSBC-treated (E0) plants are hereafter called W1 and E1 seeds, respectively]. W1 and E1 plants germinated from W1 and E1 seeds were grown under greenhouse conditions and then exposed to drought for soybean and common bean plants to examine the memory effect of MSBC treatment on drought tolerance in second generation plants. FIGS. 9A-9D and 10A-10B showed enhanced drought tolerance of E1 plants in both species, indicating the existence of transgenerational memory imprinted by MSBC treatment. Applicant observed that E1 soybean plants showed better drought tolerance than W1 (FIGS. 9A-9B) and lower ROS accumulation (FIGS. 9C-9D) under drought conditions. The E1 common bean plants showed better drought tolerance and photosynthetic performance than W1 under drought conditions (FIGS. 10A-10B).

### Example 1.6. Ethanol Treatment Affects Expression of Genes Involved in Regulating DNA Methylation in Arabidopsis thaliana

To investigate the effects of ethanol treatment on expression level of genes involved in regulation of DNA methylation, Applicant reanalyzed the microarray of Arabidopsis seedlings treated with ethanol. As summarized in Table 1, Applicant found the significant downregulation of the expression of MET1, FDA13, IDM3, HAT8 and HAT11 under ethanol treatment. Reduced expression of these genes could repress the DNA methylation of the downstream target genes which might contribute to enhance salt tolerance in Arabidopsis seedlings. In addition, Applicant found expression of RDM1 was increased by ethanol treatment in both Arabidopsis (Table 1). RDM1 is a part of a DDR complex (formed with DEFECTIVE IN MERISTEM SILENCING (DMS3) and DEFECTIVE IN RNA-DIRECTED DNA METHYLATION 1 (DRD1)), which is required for polymerase V transcripts and RNA-directed DNA methylation. These data indicated that ethanol treatment could alter the expression of genes involved in the regulation of DNA methylation in Arabidopsis thaliana.

TABLE 1
Expression of several genes involved in the regulation
of DNA methylation in Arabidopsis thaliana.

| | Ethanol control versus | | |
|---|---|---|---|
| | Water control | | |
| | | FDR- | |
| | Log2 Fold- | corrected | |
| Gene ID | change | p-value | Gene name |
| AT3G54560 | −1.0723478 | 0.000448037 | H2A.Z, HISTONE H2A |
| | | | 11, HTA11 |
| AT1G20870 | −1.001277255 | 0.001798754 | IDM3, INCREASED DNA |
| | | | METHYLATION 3 |
| AT1G63240 | −0.818532523 | 0.000714133 | RMB1, ROS1- |
| | | | ASSOCIATED METHYL- |
| | | | DNA BINDING |
| | | | PROTEIN 1 |
| AT2G17690 | −0.759594567 | 0.02682879 | ATFDA13, F-BOX/ |
| | | | DUF295 ANCESTRAL 13, |
| | | | SDC, SUPPRESSOR OF |
| | | | DRM1 DRM2 CMT3 |
| AT5G49160 | −0.689156272 | 0.000644544 | MET1 |
| AT2G38810 | −0.618845645 | 0.003372966 | H2A.Z, HISTONE H2A 8, |
| | | | HAT8 |
| AT5G59390 | 0.73702654 | 0.02317848 | FACTOR OF DNA |
| | | | DEMETHYLATION 1, |
| | | | FDDM1 |
| AT3G22680 | 0.56718444 | 1.70E−06 | RDM1, RNA-DIRECTED |
| | | | DNA METHYLATION 1 |

### Example 1.7. Summary

In this Example, Applicant showed that plant-based MSBC treatments improved abiotic stress tolerance (drought, cold and heat) in sorghum, cotton, corn, and common bean. In addition, the seeds of cotton, corn and sorghum were primed with plant-based MSBCs to test the MSBC effect on drought, heat and cold stress tolerance. Applicant observed that plant-based MSBC-treated plants (soybean, common bean, sorghum and cotton) showed lower rate of water loss and electrolyte leakage, and reduced reactive oxygen species (ROS) accumulation, but higher anthocyanin, chlorophyll and carotenoid contents than water-treated plants did under drought stress condition. These results were accompanied by higher photosynthetic performance in plant-based MSBC-treated plants compared with water-treated plants under stress conditions. Interestingly, Applicant found the ethanol-treated cotton was blooming earlier than water-treated plants. In addition, Applicant found the ethanol-treated plants had higher seed yield as compared with water-treated plants (common bean) under drought stress condition.

Next, the first-generation (soybean, common bean, cotton, sorghum, and corn) plants were treated with MSBCs (Applicant first tested with two plant-based chemicals, namely acetic acid and ethanol) or control. Thereafter, Applicant continued to grow under normal conditions in a greenhouse to harvest the next generation seeds (chemical treated seeds are hereafter called E1, and control-treated seeds are hereafter called W1).

The second-generation plants were grown under greenhouse conditions and then exposed to drought stress at different developmental stages to examine the chemical-mediated transgenerational memory effects on stress tolerance and plant productivity. Applicant's results showed that priming the first-generation plants with MSBCs enhanced stress tolerance in cotton, sorghum, soybean and common bean in the second-generation.

Applicant also found that the MSBC-treated plants had higher seed yield as compared with water-treated plants of second-generation soybean under drought stress condition. Additionally, Applicant observed that MSBC-mediated transgenerational memory effects reduced reactive oxygen species (ROS) accumulation and maintained photosynthetic performance under stress condition in the second-generation. To test how the memory effects of plant-based MSBC treatment improved plant abiotic stress tolerance and plant sustainability. Applicant analyzed several stress responses-related physiological and biochemical parameters in the second-generation plants in cotton, sorghum, soybean and common bean under normal and drought stress. Applicant observed that E1 plants showed reduced reactive oxygen species (ROS) accumulation than W1 plants did under drought stress condition. These results were accompanied by higher photosynthetic performance in E1 plants compared with W1 plants, under stress conditions. These results collectively demonstrate that the transgenerational effects of MSBC treatment on improved photosynthetic performance, and antioxidant defense, resulting in enhanced stress tolerance of next-generation plants.

To investigate the effects of MSBC treatment on expression level of genes involved in regulation of epigenetic modifications, Applicant reanalyzed the available microarray and RNA-sequence data of plants treated with ethanol and found that ethanol treatment altered the expression of important genes, which are involved in regulation of DNA methylation. These data indicate that the plant-based MSBC-mediated transgenerational memory effect on plant sustainability and plant stress tolerance could depend on epigenetic modifications.

Plant (crop) yield is severely affected by abiotic stresses, especially at seedlings and flowering stages. To estimate the memory effect of MSBC treatment on plant productivity under abiotic stress, the total yield per plants was measured in greenhouse condition. Plant (common bean and sorghum) yield was tested under normal and drought stress condition to examine the potential application of plant-based MSBC-induced epigenetic modification in agricultural productions. Impressively, stress-exposed plants showed reduced yield losses caused by drought stress. These results indicate that plant-based MSBC-treatment could rescue plant yield loss caused by abiotic stress conditions.

In summary, Applicant's results are the first findings of the trans-generational memory effects of MSBC treatment on enhanced abiotic stress tolerance and maintained plant sustainability in plants. The selected plant-based MSBC treatment and the plant developmental stage are unique, and are being reported for the first time.

Importantly, the second-generation of MSBC-treated plants (E1) showed enhanced stress tolerance and maintained productivity under stress condition without chemical treatment, suggesting a potential agricultural application to develop the stress-tolerant and non-GMO crops. In addition, the higher photosynthetic performance could contribute to maintaining plant productivity, suggesting that a potential agricultural technology could be developed for reduced water consumption in crops.

Advantageously, the seed materials are completely organic and can immediately be used without any restriction. Additionally, the collected chemicals are cost-effective and commercially available. Moreover, the plant-based MSBCs are environmental-friendly. Accordingly, the plant-based MSBC treatments maintain plant sustainability and enhance abiotic stress tolerance in the first- and the second-generation in plants via inherited epigenetic effect.

### Example 2. Effects of Ethanol and Acetic Acid on Plant Growth and Development in Sorghum

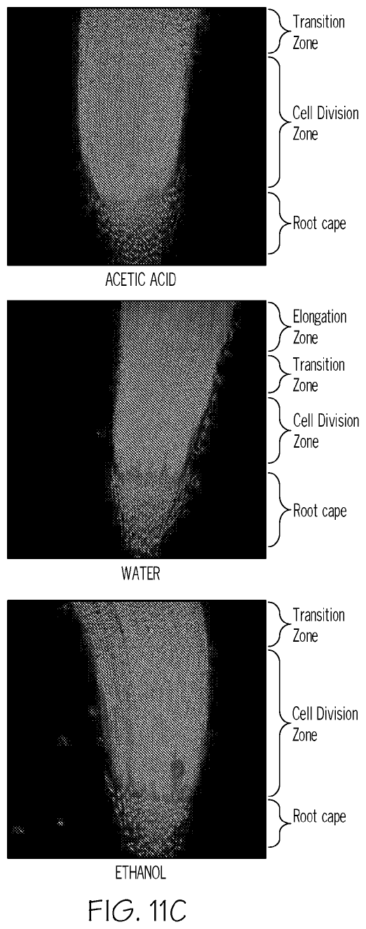

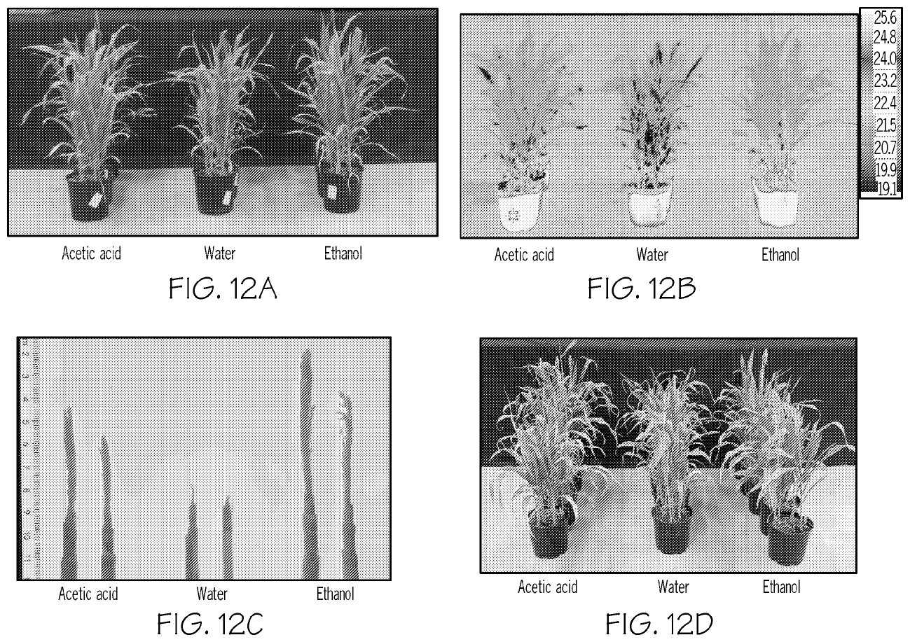

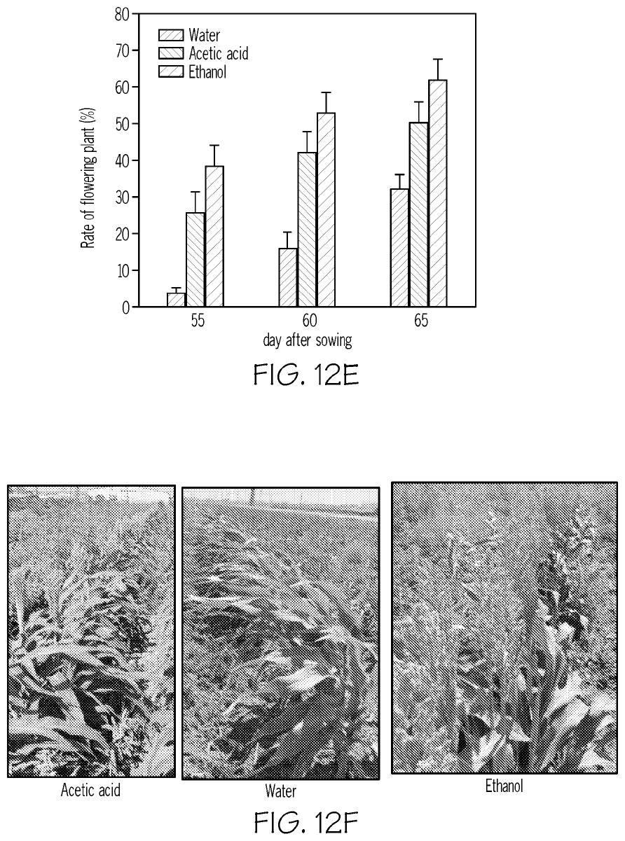

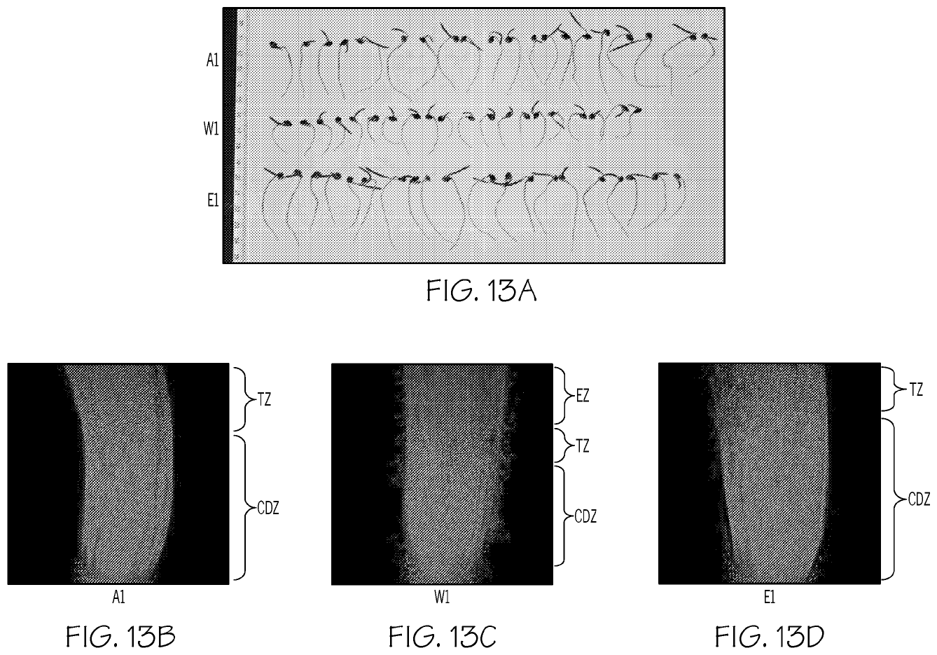

In this Example, Applicant shows that acetic acid or ethanol improved root growth (FIGS. 11A-11C) and shoot growth (FIGS. 12A-12F) in sorghum. In particular, Applicant observed that plants germinated from seeds primed with acetic acid or ethanol showed longer primary root (FIGS. 11A-11B) than water control plants under normal growth conditions. These results were accompanied by longer transition and cell division zone of the root tip of the primary root of plant-based MSBC-seed-primed plants compared with water control plants (FIG. 11C). The results suggested that acetic acid and ethanol could increase root cell division in sorghum.

Similarly, Applicant found the plant-based MSBC-treated plants grew faster (FIG. 12A), and had lower leaf temperature (FIG. 12B), faster inflorescence formation (FIG. 12C), and earlier blooming in the greenhouse (FIGS. 12D-12E) and field growth conditions (FIG. 12F) than water-treated plants. These collective results indicate that acetic acid and ethanol stimulated plant growth and development in sorghum.

### Example 2.1. Acetic Acid- and Ethanol-Mediated Transgenerational Memory Effects on Stimulating Plant Growth and Development of the Second-Generation Sorghum

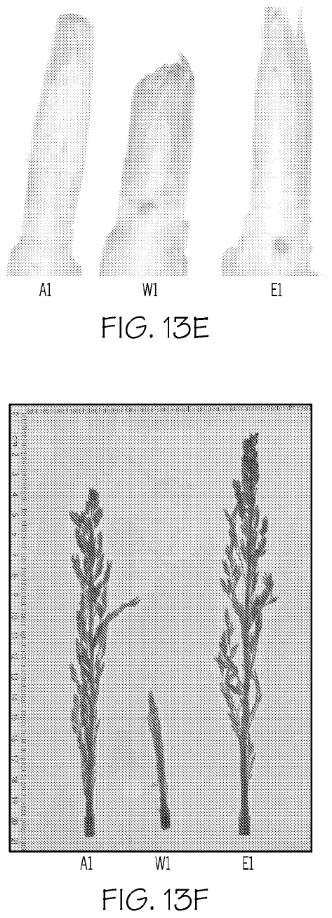

In this Example, Applicant demonstrate that plant-based MSBC (acetic acid or ethanol) memorized its effects on stimulating primary root growth, inflorescent development and blooming in the second-generation sorghum (FIGS. 13A-13F). The second-generation (progeny) sorghum seeds were collected from the plants germinated from seeds primed with acetic acid (20 mM) or ethanol (50 mM) or water (control) for 16 hours and grown under normal irrigation conditions in the greenhouse until harvest [seeds harvested from water-primed (W0), acetic acid-primed (A0), and ethanol-primed (E0) plants are hereafter called W1, A1 and E1 seeds, respectively]. Similar as the first generation. Applicant observed that the second-generation plant-based MSBC-treated (A1 and E1) sorghum plants showed longer primary root growth with longer transition and cell division zone of root tip (FIGS. 13A-13D), and faster inflorescence development (FIGS. 13E-13F) than the second-generation control (W1) plants. These results indicate that plant-based MSBC (acetic acid or ethanol) memorized its effects on stimulating plant growth and development in the second-generation sorghum.

### Example 2.2. Acetic Acid and Ethanol Enhanced Abiotic Stress Tolerance in Sorghum

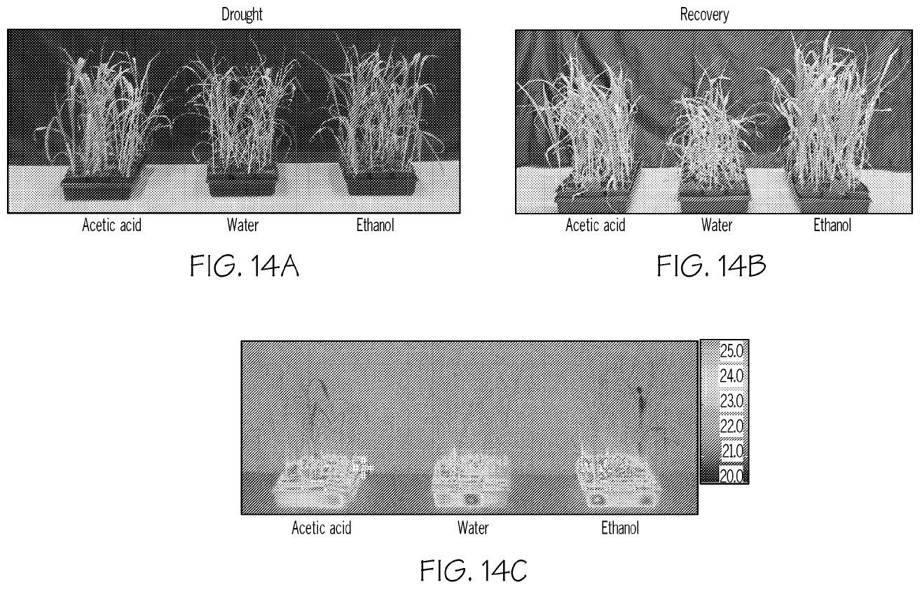

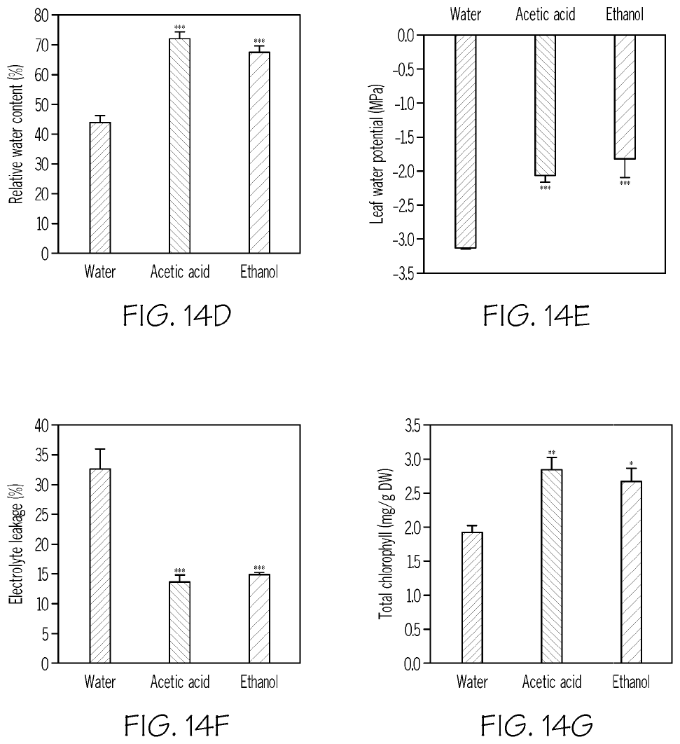

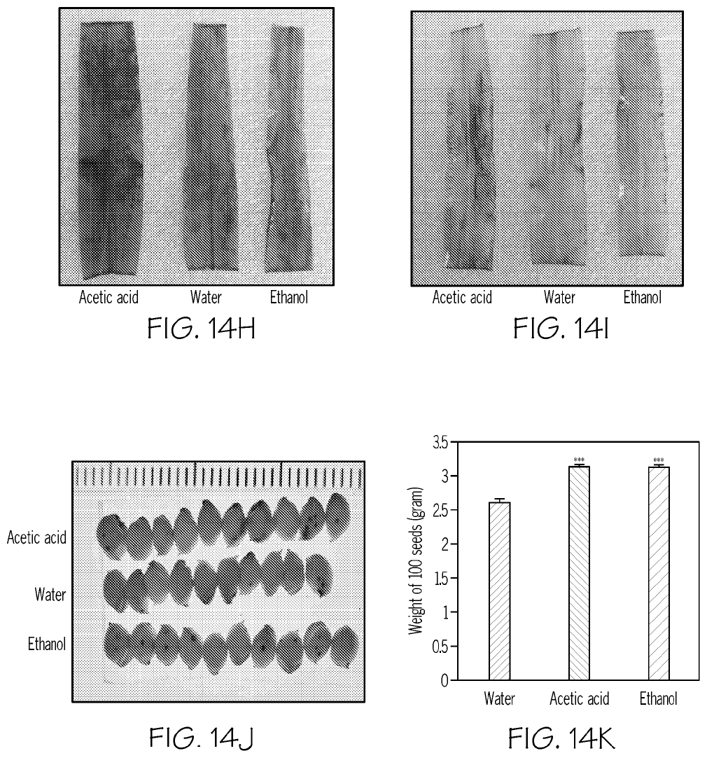

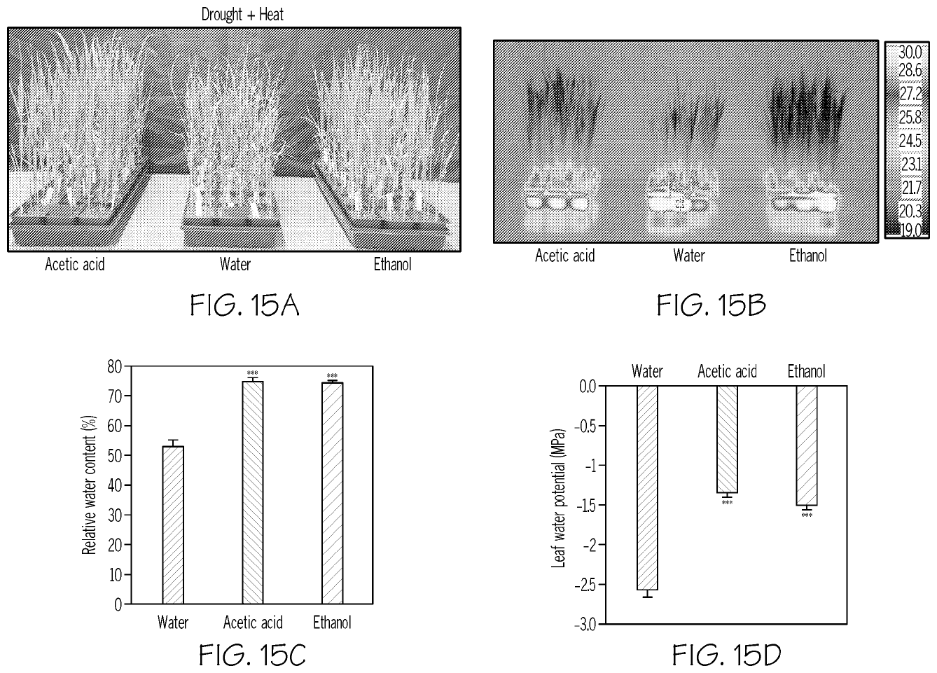

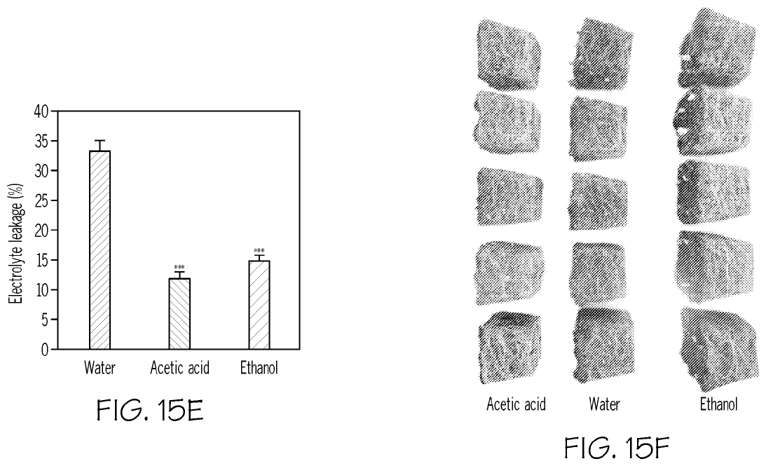

In this Example, Applicant showed that plant-based MSBC (acetic acid or ethanol) enhanced drought tolerance (FIGS. 14A-14K), and combined drought and heat stress tolerance (FIGS. 15A-15F) in sorghum. In particular, under drought stress conditions, Applicant observed that acetic acid- or ethanol-seed-primed plants showed heathier plants (FIG. 14A), better drought recovery (FIG. 14B), lower leaf temperature (FIG. 14C), higher relative leaf water content and leaf water potential (FIGS. 14D-14E), lower electrolyte leakage (FIG. 14F), higher total chlorophyll content (FIG. 14G), and reduced reactive oxygen species (ROS) (e.g. superoxide and hydrogen peroxide) accumulation (FIGS. 14H-14I) than water-seed-primed plants.

In addition, the acetic acid- or ethanol-seed-primed plants showed bigger seed size and greater weight of 100 seeds than water-seed-primed plants under normal growth condition (FIGS. 14J-K). Similarly, Applicant observed that acetic acid- or ethanol-seed-primed plants showed better combined drought and heat tolerance than water-seed-primed plants, indicated by heathier plants (FIG. 15A), lower leaf temperature (FIG. 15B), higher relative leaf water content and leaf water potential (FIGS. 15C-15D), lower electrolyte leakage (FIG. 15E), and more roots (FIG. 15F) under combined drought and heat stress conditions.

### Example 2.3. Acetic Acid- and Ethanol-Mediated Transgenerational Memory Effects on Drought, and Combined Drought and Heat Tolerance of the Second-Generation Sorghum

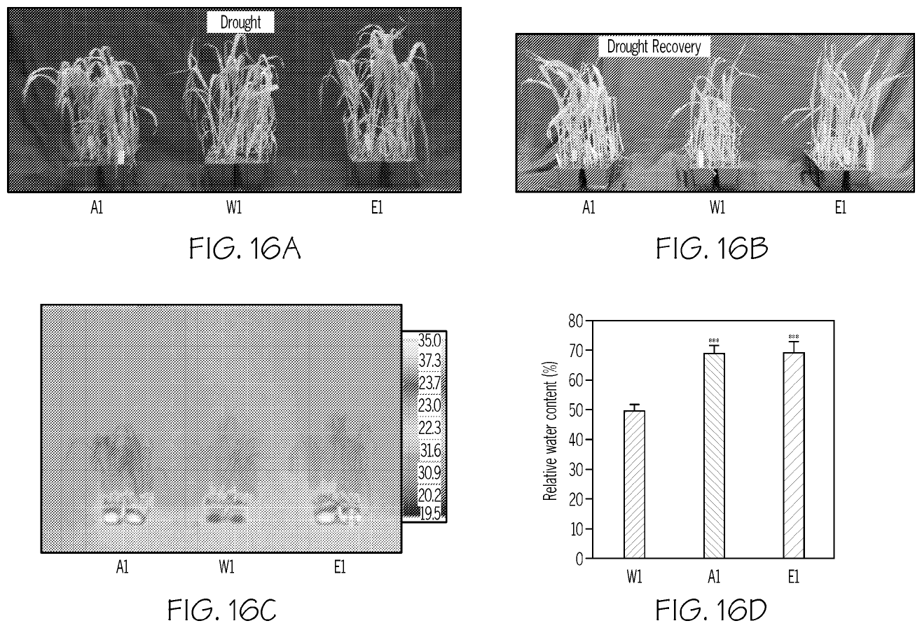

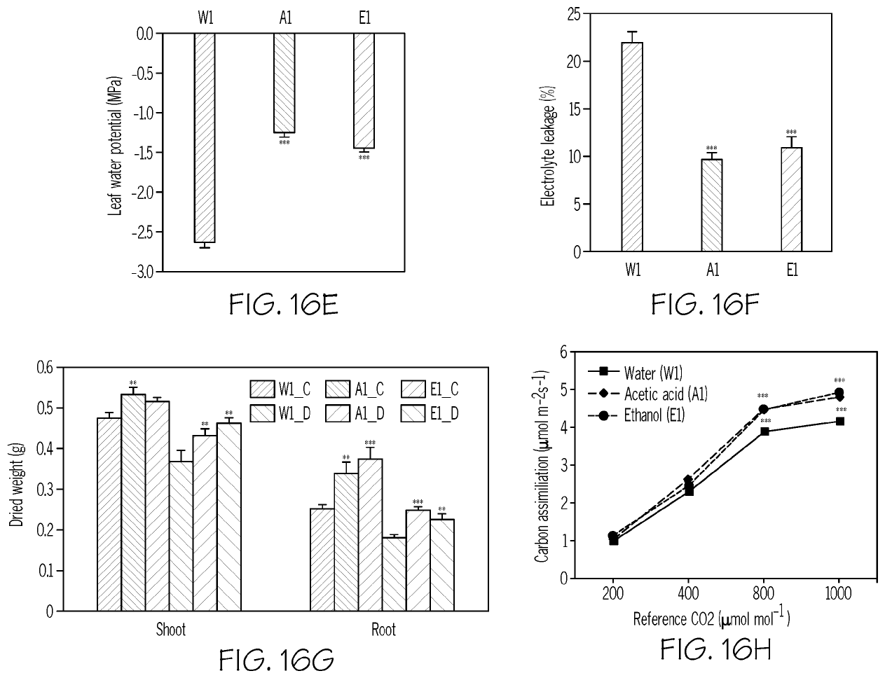

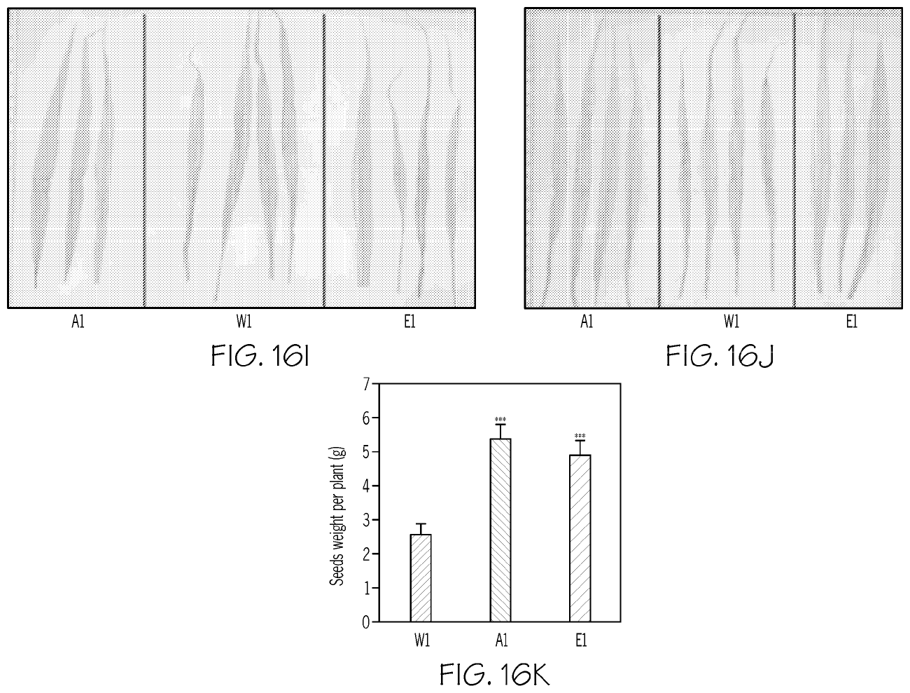

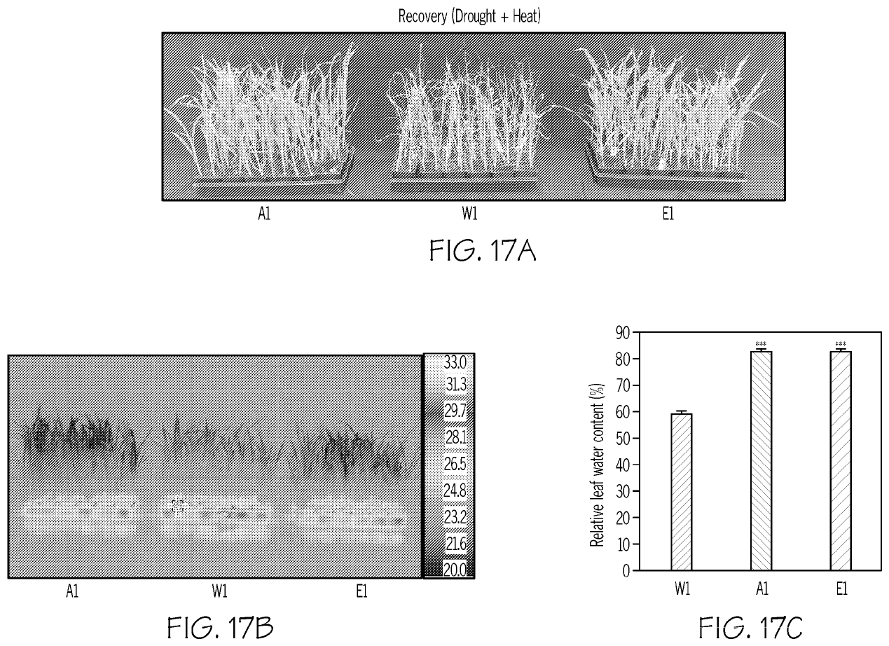

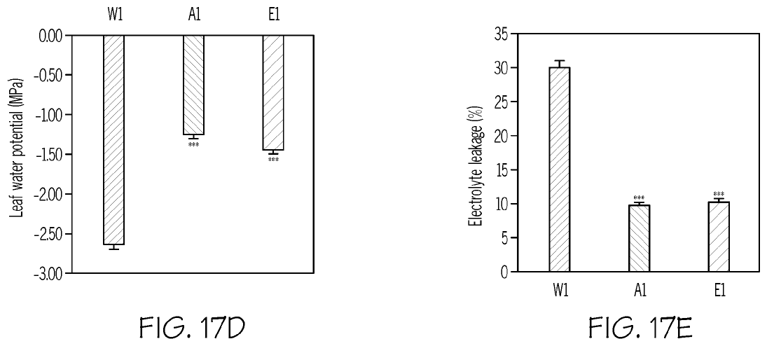

In this Example, Applicant showed that plant-based MSBC (acetic acid or ethanol) memorized its effects on enhancing drought (FIGS. 16A-16K), and combined drought and heat (FIGS. 17A-17E) tolerance of the second-generation sorghum. The second-generation sorghum seeds were collected from the plants germinated from seeds primed with acetic acid (20 mM) or ethanol (50 mM) or water (control) for 16 hours and grown under normal condition in the greenhouse until harvest [seeds harvested from water-primed (W0), acetic acid-primed (A0), and ethanol-primed (E0) plants are hereafter called W1, A1 and E1 seeds, respectively].

Under drought conditions, Applicant observed that the second-generation plant-based MSBC-treated (A1 and E1) sorghum plants showed heathier plants (FIG. 16A), better drought recovery (FIG. 16B), lower leaf temperature (FIG. 16C), higher relative leaf water content and leaf water potential (FIGS. 16D-16E), lower electrolyte leakage (FIG. 16F), higher shoot and root biomass (FIG. 16G), better photosynthetic performance (FIG. 16H), lower ROS (e.g. superoxide and hydrogen peroxide) accumulation (FIGS. 16I-J), and greater seed weight per plants (FIG. 16K) than W1 plants.

Similarly, Applicant observed the enhancement of combined drought and heat tolerance in the second-generation A1 and E1 plants indicated by better recovery (FIG. 17A), lower leaf temperature (FIG. 17B), higher relative leaf water content and leaf water potential (FIGS. 17C-17D), and lower electrolyte leakage (FIG. 17E) than W1 plants. These collective results indicated that plant-based MSBC (acetic acid and ethanol) memorized its effects on enhancing drought, and combined drought and heat tolerance of the second-generation sorghum.

### Example 2.4. Root Growth Assay

The sorghum seeds (BTX623) were treated with acetic acid (20 mM) or ethanol (50 mM) or water for 16 hours in a 50 mL falcon tube. After 16 hours of soaking, the solutions were discarded from the tube and seeds were kept in the tube for 3 days to germinate. The primary root length of 3-day-old plants was measured.

For confocal microscopic analysis, to stop the root growth, the roots of 3-day-old plants were fixed in solutions containing 95% ethanol and glacial acetic acid (3:1 ratio) for overnight. The root samples were then performed cleaning and staining before observing under confocal microscope.

For root cleaning assay, the roots were cut and put in a petri dish containing 0.8M NaOH solution for overnight. For root staining assay, root samples were placed in petri dish containing propidium iodide (1 g in 10 mL 1× phosphate buffered saline (PBS) buffer) overnight under conditions. The root samples (1 cm root tip portion was placed on groove containing 50% glycerol) were then observed using confocal microscope.

### Example 2.5. Shoot Growth Assay in the Greenhouse and Field Conditions

The sorghum seeds (BTX623) were treated with acetic acid (20 mM) or ethanol (50 mM) or water for 16 hours. For the greenhouse experiment, the primed-seeds were shown in 7.6-L plastic pots containing BM7 soil (Berger, Canada) and grown under normal irrigation conditions in the greenhouse. The fluorescent development and blooming were recorded. The second-generation seeds were collected for further studies. For the field experiment, the primed-seeds were planted in the Quaker Research Farm (Texas Tech University) field and the blooming were recorded.

### Example 2.6. Plant Growth and Stress Treatment

Sorghum seeds (BTX623) were treated with acetic acid (20 mM) or ethanol (50 mM) water for 16 hours. The primed-seeds were sown in 0.7-L plastic pots containing BM7 soil (Berger, Canada) and grown in the greenhouse for 21 days. For drought treatment, 21-day-old plants were withheld from water for 7 days, then rewatered for 5 days. For combined drought and heat stress treatment, 21-day-old plants were withheld from water in growth chamber (40° C., 800 μmol m⁻² s⁻¹ photon flux density, 50% relative room humidity) for 7 days, then recovery for 5 days.

### Example 2.7. Determination of Relative Water Contents, Leaf Water Potential, Electrolyte Leakage, Leaf Surface Temperature, Total Chlorophyll Content, ROS Staining Assay, Shoot and Root Dried Weight

On the 6^{th} days after drought stress or the 5^{th} days after combined drought and heat stress, the second leaves (counted from top) were sampled, and the relative water content, and electrolyte leakage were measured. The leaf water potential was measured using the PMS 1515D system (PMS Ins. Co, USA). Thermal images to detect leaf surface temperature were taken using an InfReC R450Pro camera (Nippon Avionics Co., Ltd., Japan). The total chlorophyll contents were measured using spectrometer machine at 645 and 663 nm. The ROS staining assay to detect superoxide (using nitroblue tetrazolium staining) and hydrogen peroxide (using 3,3-diaminobenzidine staining) was performed. For measuring shoot and root dried weight, the shoot and root samples were separated and dried for 3 days at 72 degrees C.

### Example 2.8. Determination of Photosynthetic Activity

Photosynthetic parameters were determined using a LI-6800 photosynthesis system (LI-COR Biosiences, USA). Photosynthetic activities of the second leaf (counted from top) were measured on the 6^{th} day after starting drought treatment. The instantaneous water-use-efficiency (WUEᵢₙₛ) was determined by the ratio between the CO₂ assimilation and the transpiration rates.

### Example 3. Effects of Ethanol and Acetic Acid in Cotton and Common Bean

In this Example, Applicant describes the identification of 2 MSBCs (natural plant-based chemicals namely acetic acid and ethanol) enhanced abiotic stress tolerance in cotton and common bean plants. In particular, Applicant found plant-based MSBC (ethanol and acetic acid) enhanced drought tolerance of cotton and common bean, and enhanced heat tolerance in common bean in the first-generation. Importantly, Applicant found that memory effect of plant-based MSBC (ethanol and acetic acid) treatments enhanced drought tolerance in the second-generation cotton and common bean, and enhanced heat tolerance in the second-generation common bean. These data suggested the existence of inherited memory effects of these plant-based MSBCs on abiotic stress tolerance across generations in cotton and common bean plants.

### Example 3.1. Acetic Acid and Ethanol Enhanced Abiotic Stress Tolerance in Cotton and Common Bean

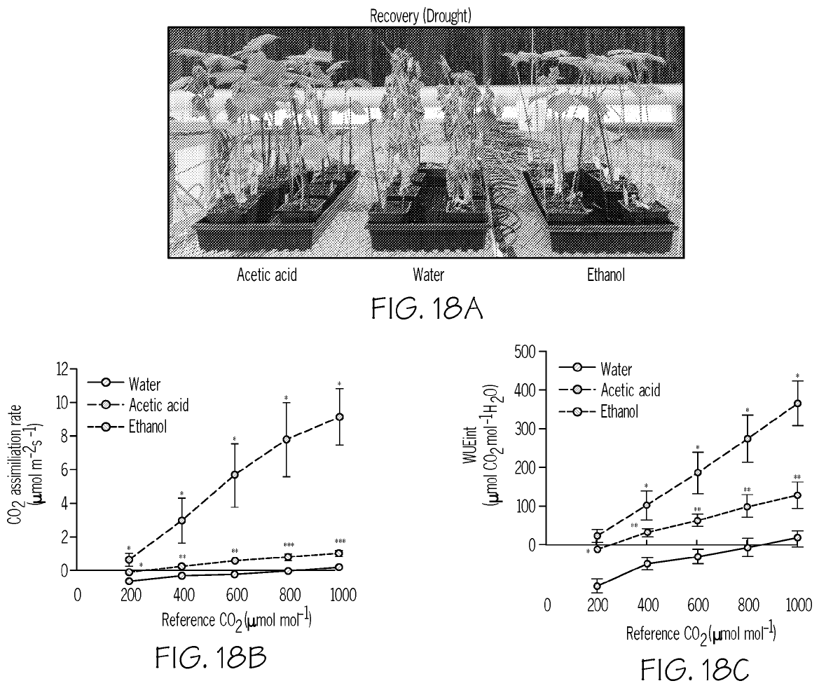

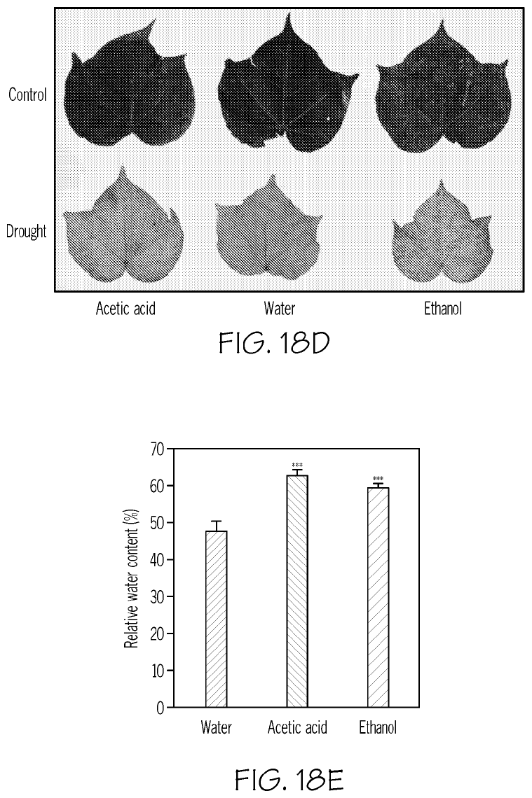

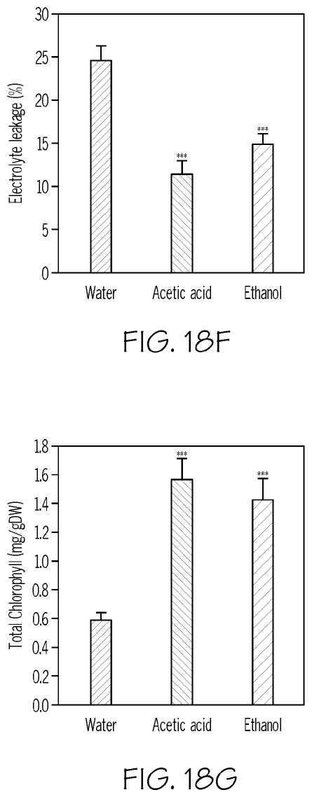

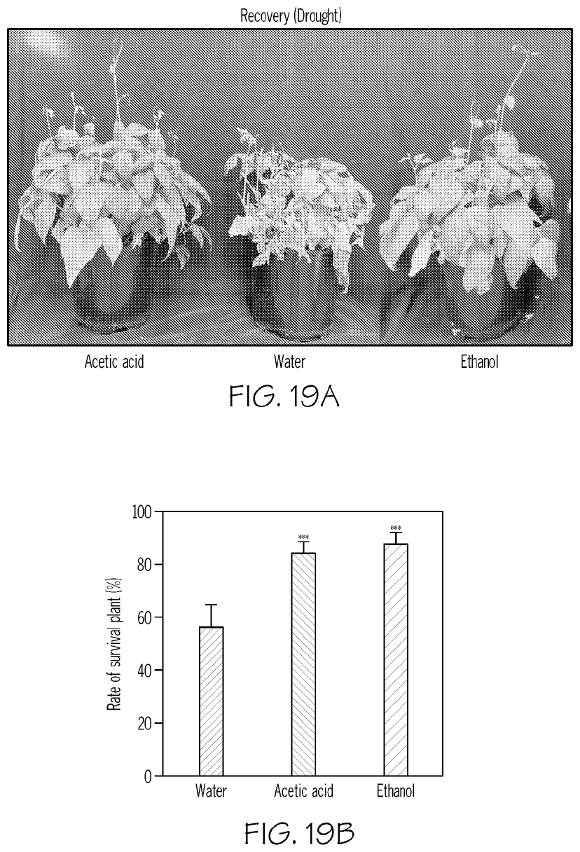

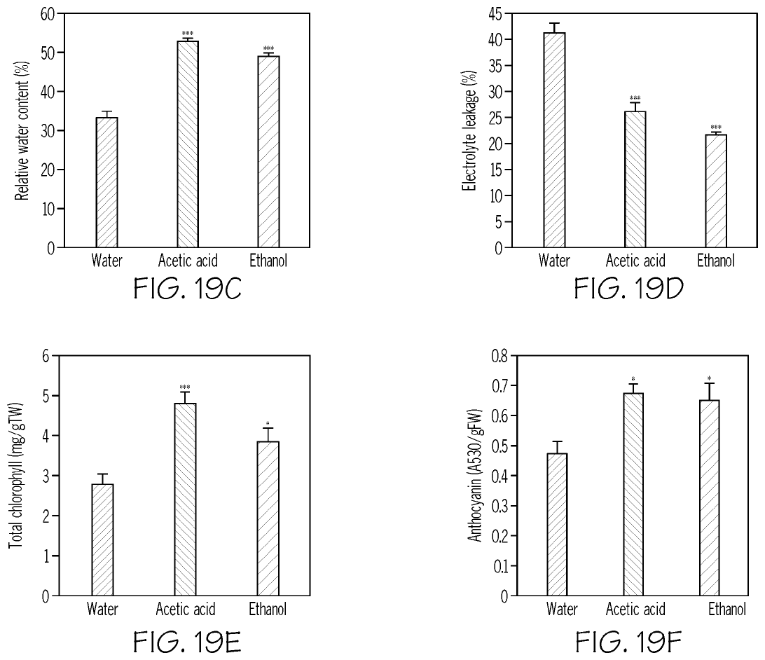

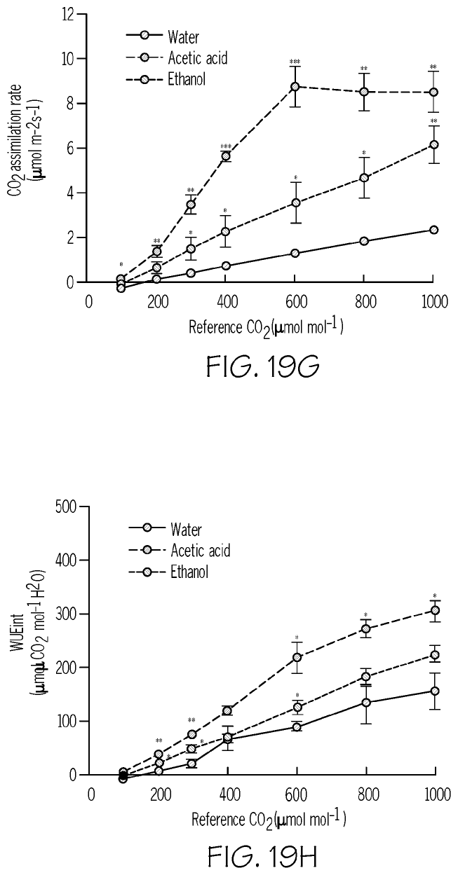

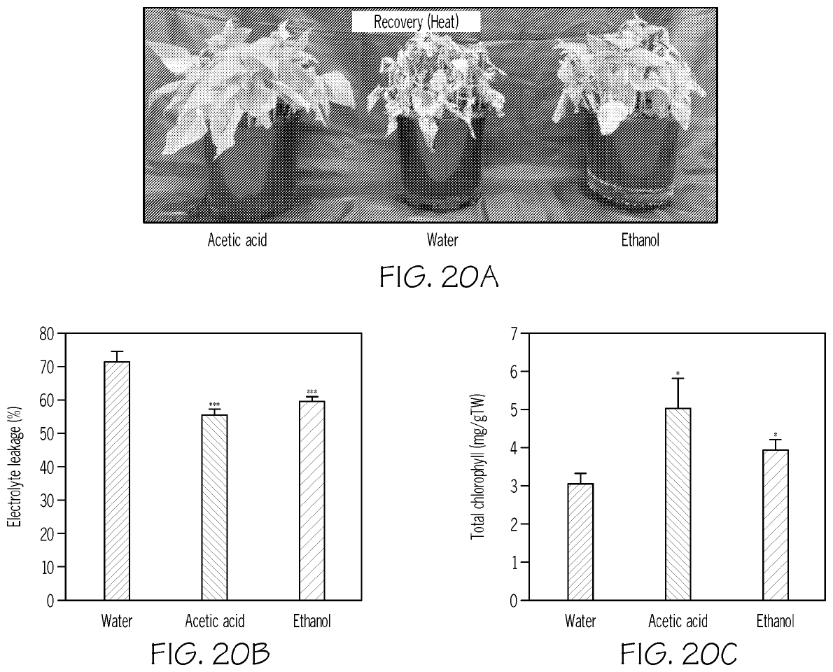

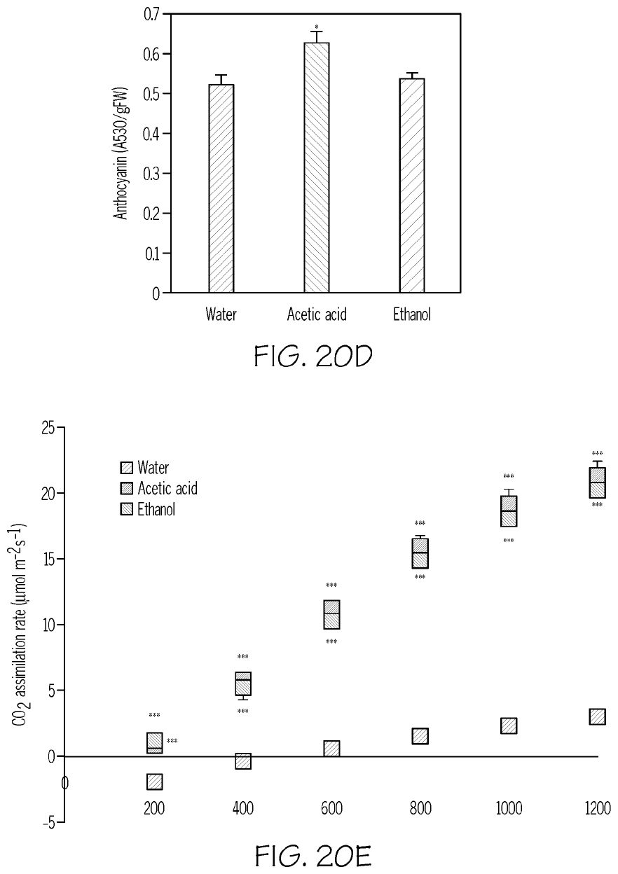

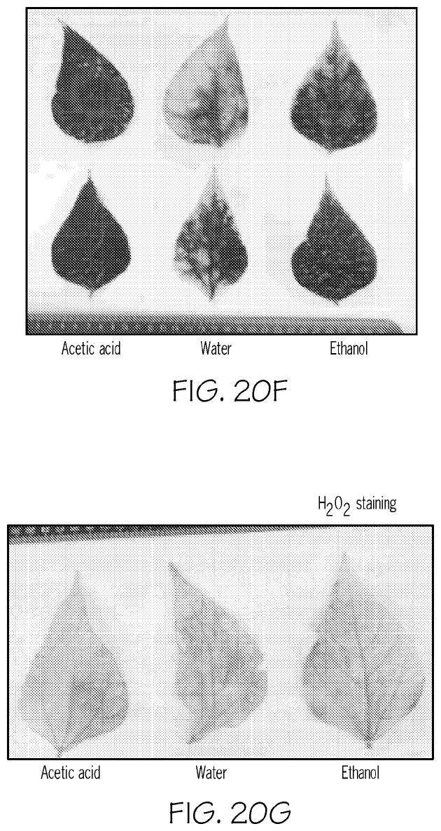

In this Example, Applicant demonstrates that acetic acid and ethanol enhanced drought tolerance in cotton (FIGS. 18A-18G) and common bean (FIGS. 19A-19H), and heat tolerance in common bean (FIGS. 20A-20G). In particular, under drought stress condition. Applicant observed that acetic acid- or ethanol-treated cotton plants showed better drought recovery (FIG. 18A), better photosynthetic performance indicated by higher CO₂ assimilation rate (FIG. 18B), instantaneous water-use-efficiency (FIG. 18C), greater starch accumulation (FIG. 18D), higher relative leaf water content (FIG. 18E), lower electrolyte leakage (FIG. 18F), and higher total chlorophyll content (FIG. 18G) than water-treated plants.

Similarly, Applicant found better drought tolerance of acetic acid- or ethanol-treated common bean (FIGS. 19A-19H), as indicated by better drought recovery (FIGS. 19A-19B), higher relative leaf water content (FIG. 19C), lower electrolyte leakage (FIG. 19D), and higher total chlorophyll (FIG. 19E) and anthocyanin (FIG. 19F) contents, and better photosynthetic performance indicated by higher CO₂ assimilation rate (FIG. 19G) and water-use-efficiency (FIG. 19H) than water-treated plants under drought conditions.

In response to heat stress of common bean, Applicant observed that the acetic acid- or ethanol-treated plants exhibited better heat tolerance than water-treated plants (FIGS. 20A-20G). Applicant found that acetic acid- or ethanol-treated plants showed better heat recovery (FIG. 20A), lower electrolyte leakage (FIG. 20B), higher total chlorophyll (FIG. 20C) and anthocyanin content (FIG. 20D), and better photosynthetic performance (FIG. 20E), more starch accumulation (FIG. 20F), and less accumulation of reactive oxygen species (e.g. hydrogen peroxide) (FIG. 20G) than water-treated plants under heat stress conditions. These collective results indicate that acetic acid and ethanol enhanced drought tolerance in cotton and common bean, and heat tolerance in common bean.

### Example 3.2. Acetic Acid- and Ethanol-Mediated Transgenerational Memory Effects on Drought Tolerance of the Second-Generation Cotton

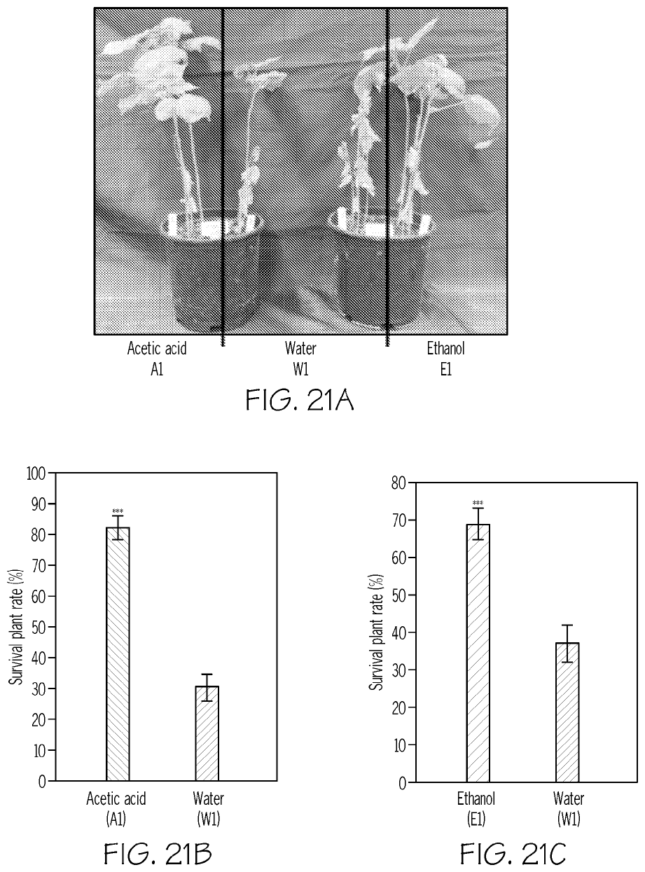

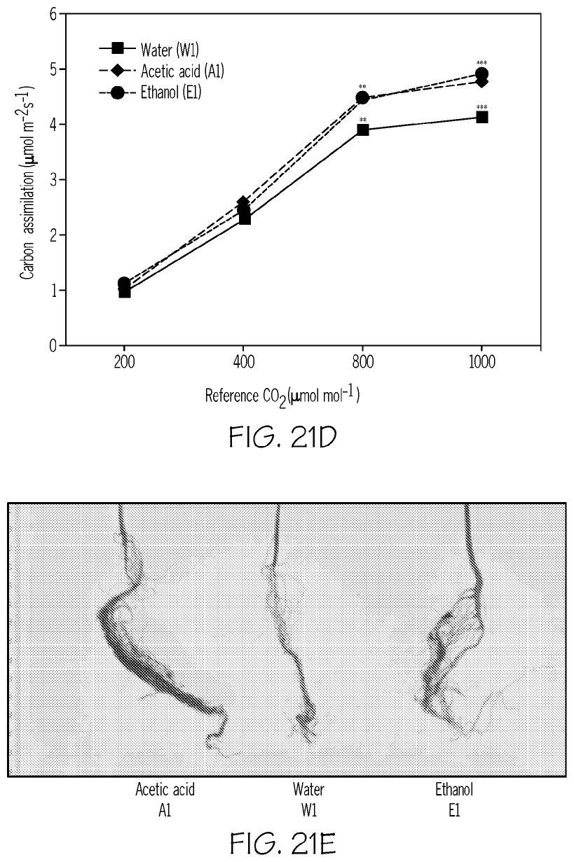

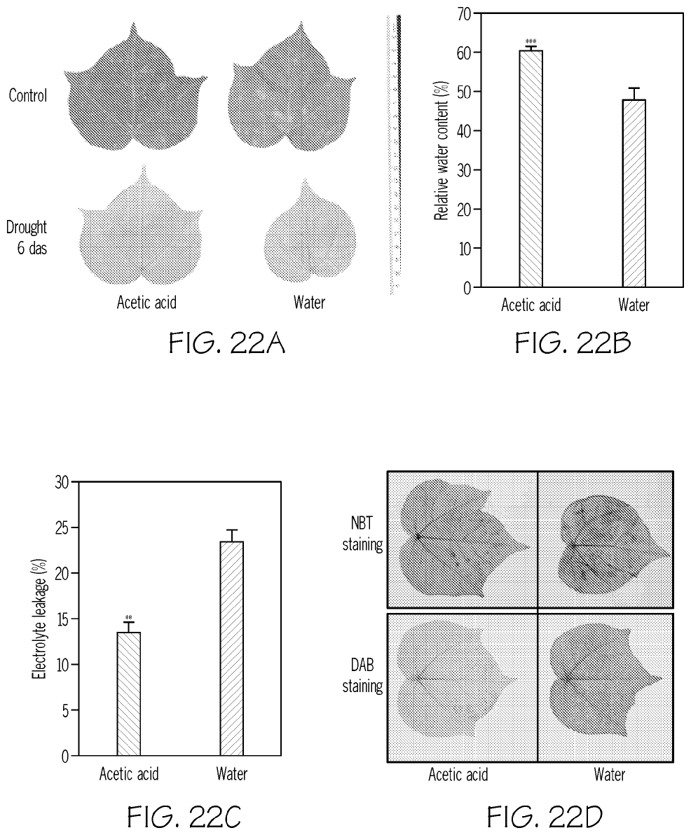

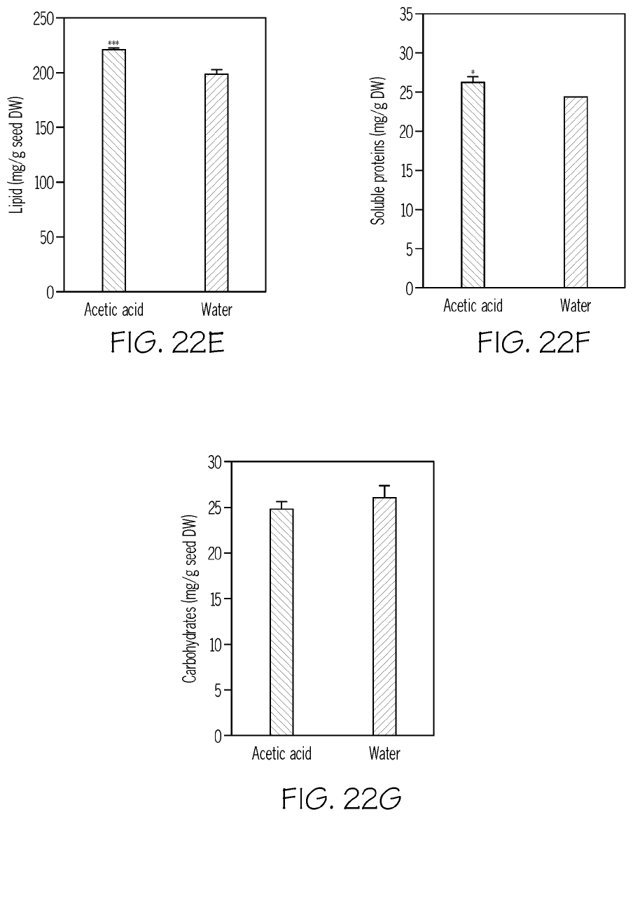

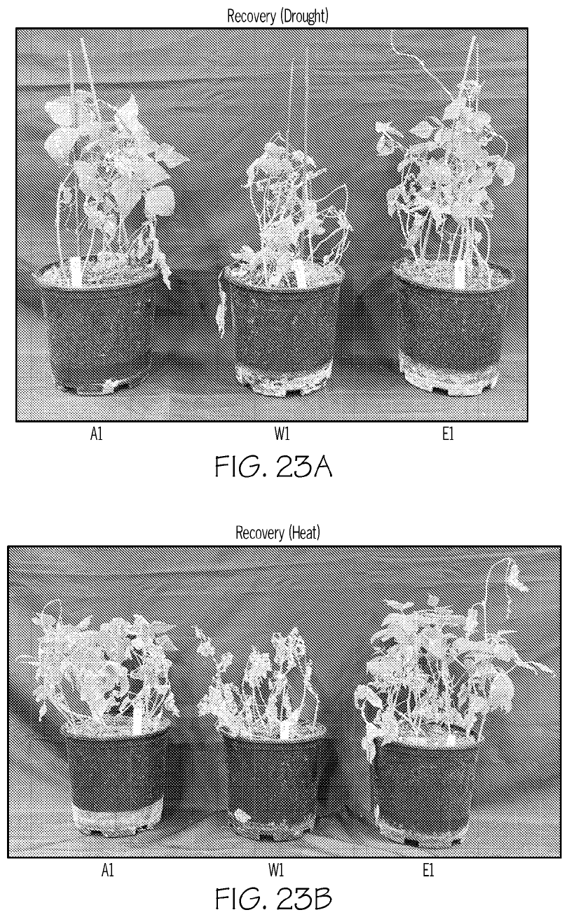

To investigate whether acetic acid induces memory effect leading to drought tolerance of second-generation cotton plants, 35-day-old cotton plants were treated with water (control) or acetic acid (20 mM) or ethanol (50 mM) (supplemented to soil) for 2 days, and then continued to grow under non-stressed conditions until harvest [seeds harvested from water-treated (W0), acetic acid-treated (A0) plants, and ethanol-treated (A0) plants are hereafter called W1, A1 and E1 seeds, respectively]. W1, A1 and E1 plants germinated from W1, A1 and E1 seeds were grown under green conditions for 21 days and then exposed to drought for 12 days to examine the acetic acid- and ethanol-mediated transgenerational memory effects on drought tolerance in second-generation plants. Data revealed that A1 and E1 plants had increased drought tolerance (FIGS. 21A-21E) as indicated by higher survival rate after drought recovery (FIGS. 21A-21C), better photosynthetic performance (FIG. 21D), and greater root biomass (FIG. 21E) than W1 plants under drought conditions. Applicant also found that A1 plants had greater starch accumulation (FIG. 22A), higher leaf relative water content (FIG. 22B), better cell membrane integrity (FIG. 22C), and lower reactive oxygen species (ROS) (e.g. superoxide and hydrogen peroxide) accumulation (FIG. 22D) under drought conditions in the greenhouse experiment. In addition, the A1 seeds showed greater lipid (oil) (FIG. 22E) and soluble protein (FIG. 22F) and unchanged carbohydrate (FIG. 22G) contents than W1 seeds. Collectively. Applicant's data indicated the existence of inherited memory effects of acetic acid and ethanol on drought tolerance across generations in cotton.

### Example 3.3. Acetic Acid and Ethanol Memorized its Effects on Enhancing Abiotic Stress Tolerance in the Second-Generation Common Bean

In this Example, Applicant showed that plant-based MSBC (acetic acid or ethanol) memorized its effects on enhancing drought and heat tolerance of the second-generation common bean (FIGS. 23A-23B). The second-generation cotton and common bean seeds were collected from the plants germinated from the 14-day-old common bean plants treated with acetic acid (20 mM) or ethanol (50 mM) or water (control) for 2 days and grown under normal condition in the greenhouse until harvest [seeds harvested from water-treated (W0), acetic acid-treated (A0), and ethanol-treated (E0) plants are hereafter called W1, A1 and E1 seeds, respectively]. Under drought conditions, Applicant observed the enhancement of drought (FIG. 23A) or heat tolerance (FIG. 23B) in the second-generation A1 and E1 common bean plants, indicated by better recovery than W1 plants. These results indicated that acetic acid and ethanol memorized its effects on enhancing drought and heat tolerance in the second-generation common bean.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of enhancing a physiological performance or environmental stress resistance of a plant or seed, said method comprising:
exposing the plant or seed to a composition,
wherein the composition comprises one or more active ingredients selected from the group selected from the group consisting of ethanol, acetic acid, and combinations thereof.

2. The method of claim 1, wherein the exposing enhances the physiological performance of the plant or seed relative to an untreated plant or seed, respectively.

3. The method of claim 2, wherein the enhanced physiological performance is selected from the group consisting of reduced water consumption, enhanced photosynthetic performance, an increase in number of photosynthetic pigments, enhanced antioxidant defense, enhanced antioxidant accumulation, enhanced flowering, enhanced seed maturity, enhanced growth, an increase in soluble proteins, an increase in starch, increased seed yield, a reduction in water loss, reduced electrolyte leakage, a reduction in reactive oxygen species (ROS) accumulation, lower malondialdehyde accumulation, enhanced root growth, enhanced shoot growth, reduced leaf temperatures, and combinations thereof.

4. The method of claim 2, wherein the enhanced physiological performance is inheritable in the plant or seed, and wherein the offspring plants or seeds from the treated plant or seed demonstrate substantially the same enhanced physiological performance as the treated plant or seed, respectively.

5. The method of claim 1, wherein the exposing enhances the resistance of the plant or seed to one or more environmental stresses relative to an untreated plant or seed, respectively.

6. The method of claim 5, wherein the one or more environmental stresses are selected from the group consisting of drought, heat, freezing temperatures, microbial contamination, biotic stress, abiotic stress, plant pathogenesis, and combinations thereof.

7. The method of claim 5, wherein the enhanced environmental stress resistance is inheritable in the plant or seed, and wherein the offspring plants or seeds from the treated plants or seeds demonstrate substantially the same resistance to the one or more environmental stresses as the treated plant or seed, respectively.

8. The method of claim 1, wherein the treated plant or seed is selected from the group consisting of maize, rice, bean, soybean, common bean, pinto bean, corn, cotton, wheat, N. benthamiana, Arabidopsis, tobacco, tomato, lettuce, potato, grapes, sorghum, varieties thereof, and combinations thereof.

9. The method of claim 1, wherein the treated plant or seed is selected from the group consisting of soybean, common bean, pinto bean, corn, cotton, Arabidopsis, sorghum, varieties thereof, and combinations thereof.

10. The method of claim 1, wherein the treated plant or seed comprises sorghum.

11. The method of claim 1, wherein the treated plant or seed comprises a treated plant.

12. The method of claim 11, further comprising a step of collecting offspring seeds from the treated plant and growing offspring plants from the offspring seeds.

13. The method of claim 12, wherein the enhanced environmental stress resistance is inheritable in the offspring plants, and wherein the offspring plants demonstrate substantially the same resistance to one or more environmental stresses as the treated plant.

14. The method of claim 12, wherein the enhanced physiological performance is inheritable in the offspring plants, and wherein offspring plants demonstrate substantially the same enhanced physiological performance as the treated plant.

15. The method of claim 1, wherein the treated plant or seed comprises a treated seed.

16. The method of claim 15, further comprising a step of germinating the treated seeds to produce offspring plants from the treated seeds.

17. The method of claim 15, wherein the offspring plants demonstrate enhanced physiological performance relative to an untreated plant.

18. The method of claim 15, wherein the offspring plants demonstrate enhanced resistance to one or more environmental stresses relative to an untreated plant.

19. The method of claim 1, wherein the one or more active ingredients of the composition comprise ethanol.

20. The method of claim 19, wherein the concentration of the ethanol in the composition is at least about 10 mM.

21. The method of claim 19, wherein the concentration of the ethanol in the composition is at least about 50 mM.

22. The method of claim 1, wherein the one or more active ingredients of the composition comprise acetic acid.

23. The method of claim 22, wherein the concentration of the acetic acid in the composition is at least about 1 mM.

24. The method of claim 22, wherein the concentration of the acetic acid in the composition is at least about 20 mM.

25. The method of claim 1, wherein the one or more active ingredients of the composition comprise ethanol and acetic acid.

26. The method of claim 25, wherein the concentration of the ethanol in the composition is at least about 25 mM, and wherein the concentration of the acetic acid in the composition is at least about 10 mM.

27. The method of claim 25, wherein the concentration of the ethanol in the composition is at least about 50 mM, and wherein the concentration of the acetic acid in the composition is at least about 20 mM.

28. A modified plant or seed,
wherein the modified plant or seed demonstrates enhanced physiological performance, enhanced environmental stress resistance, or combinations thereof;
wherein the modified plant or seed is formed by exposing the plant or seed to a composition; and
wherein the composition comprises one or more active ingredients selected from the group selected from the group consisting of ethanol, acetic acid, and combinations thereof.

29. The modified plant or seed of claim 28, wherein the modified plant or seed demonstrates enhanced physiological performance of the plant or seed relative to an untreated plant or seed, respectively.

30. The modified plant or seed of claim 29, wherein the enhanced physiological performance is selected from the group consisting of reduced water consumption, enhanced photosynthetic performance, an increase in number of photosynthetic pigments, enhanced antioxidant defense, enhanced antioxidant accumulation, enhanced flowering, enhanced seed maturity, enhanced growth, an increase in soluble proteins, an increase in starch, increased seed yield, a reduction in water loss, reduced electrolyte leakage, a reduction in reactive oxygen species (ROS) accumulation, lower malondialdehyde accumulation, enhanced root growth, enhanced shoot growth, reduced leaf temperatures, and combinations thereof.

31. The modified plant or seed of claim 29, wherein the enhanced physiological performance is inheritable in the plant or seed.

32. The modified plant or seed of claim 29, wherein the offspring plants or seeds from the modified plant or seed demonstrate substantially the same enhanced physiological performance as the modified plant or seed, respectively.

33. The modified plant or seed of claim 28, wherein the modified plant or seed demonstrates enhanced resistance of the plant or seed to one or more environmental stresses relative to an unmodified plant or seed, respectively.

34. The modified plant or seed of claim 33, wherein the one or more environmental stresses are selected from the group consisting of drought, heat, freezing temperatures, microbial contamination, biotic stress, abiotic stress, plant pathogenesis, and combinations thereof.

35. The modified plant or seed of claim 33, wherein the enhanced environmental stress resistance is inheritable in the plant or seed.

36. The modified plant or seed of claim 35, wherein the offspring plants or seeds from the modified plants or seeds demonstrate substantially the same resistance to the one or more environmental stresses as the modified plant or seed, respectively.

37. The modified plant or seed of claim 28, wherein the modified plant or seed is selected from the group consisting of maize, rice, bean, soybean, common bean, pinto bean, corn, cotton, wheat, N. benthamiana, Arabidopsis, tobacco, tomato, lettuce, potato, grapes, sorghum, varieties thereof, and combinations thereof.

38. The modified plant or seed of claim 28, wherein the modified plant or seed is selected from the group consisting of soybean, common bean, pinto bean, corn, cotton, Arabidopsis, sorghum, varieties thereof, and combinations thereof.

39. The modified plant or seed of claim 28, wherein the modified plant or seed comprises sorghum.

40. The modified plant or seed of claim 28, wherein the modified plant or seed comprises a modified plant.

41. The modified plant or seed of claim 28, wherein the modified plant or seed comprises a modified seed.
